(19) United States
(12) Patent Application Publication
(10) Pub. No.: US 2004/0077047 A1
(43) Pub. Date: Apr. 22, 2004

(54) Method for producing heterologous proteins in a homothallic fungus of the sordariaceae family

(76) Inventors: Ulrich Kuck, Bochum (DE); Stefanie Poggeler, Bochum (DE); Sandra Masloff, Luckenwalde (DE); Minou Nowrousian, Essen (DE); Oliver Bartelsen, Koln (DE); Gerd Gellissen, Wulfrath (DE); Michael Pfeil, Bochum (DE)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/451,866

(22) PCT Filed: Dec. 28, 2001
(86) PCT No.: PCT/EP01/15354

(30) Foreign Application Priority Data
Dec. 29, 2000 (DE) 100656811
May 16, 2001 (DE) 101238576

(51) Int. Cl. C12N 1/16; C12P 21/02

(52) U.S. Cl. 435/69.1; 435/254.3

(57) ABSTRACT

The present invention relates to a method for the production of heterologous protein in a filamentous fungus and promoters suitable therefor. The present invention further relates to vectors and host cells as well as a kit and its use. The object of the present invention is to provide a method for the production of heterologous protein in a filamentous fungus which allows efficient production of heterologous protein and wherein any contamination of the product protein by vegetative spores is avoided. According to the invention this object is solved by a method for the production of heterologous protein in a filamentous fungus which comprises (a) the cultivation of a homothallic fungus of the family Sordariaceae, which contains an expression cassette which contains the following elements in functional combination: a promoter active in the fungus of the family Sordariaceae; a heterologous gene and a terminator active in the fungus of the family Sordariaceae, and (b) the harvesting of the protein produced in an inherently known fashion.

The present invention relates to a method for the production of heterologous protein in a filamentous fungus and promoters suitable therefor. The present invention further relates to vectors and host cells as well as a kit and its use.

Genetic engineering methods allow the establishment of expression systems for the production of recombinant proteins. In the approximately twenty-year tradition of industrial genetic engineering, various microorganisms or cell lines have been developed and used as host systems. The definition of a host was made according to criteria of availability, economy of a production process based thereon, the area of application and the properties of the protein to be manufactured. Various gram-positive and gram-negative bacteria, mammalian cells and different fungi including yeast and filamentous fungi have been used, among others, as expression systems.

Of particular economic importance is the manufacture of mammalian proteins, and especially human proteins, used in pharmaceutical products. Bacterial expression systems such as E. coli are frequently not suited for the production of such proteins which frequently contain a complex glycosylation pattern because bacteria do not secrete such heterologous proteins nor can they form the modifications of polypeptides associated with the secretion pathway of higher (eukaryotic) organisms, such as glycosylation. The deposition of recombinant products in inclusion bodies very frequently results in the formation of insoluble and biologically inactive protein aggregates. A heterologous product must therefore frequently be converted into a biologically active form by an expensive denaturing and renaturing method.

Thus, mammalian cells are used for the production of proteins which require a complex mammalian glycosylation. However, mammalian cell systems are very expensive and the cells are furthermore a potential target for pathogenic viruses. Yeasts and filamentous fungi, however, indeed have a glycosylation pattern which differs from the complex mammalian type, but they can be used for a plurality of products. As eukaryotic microorganisms they are capable of secretion and like bacteria they can be fermented on cheap media at high cell densities. Since the culture media contain neither serum nor other potential contaminants, the heterologous protein produced can be purified easily and cheaply. Examples of established expression systems are yeasts such as S. cerevisiae and the methylotrophic yeast Hansenula polymorpha.

Within the group of filamentous fungi regarded as better “secreters” various Aspergillus species and Neurospora crassa have been used for heterologous gene expression. However, these fungi have so far been used almost exclusively for industrial manufacture of technical enzymes because the presence of vegetative spores, which occur in large quantities in the culture and are difficult to remove, prevented them from being used for the manufacture of pharmaceutical products since spore-free preparations can only be manufactured at high cost. This particularly applies to the frequently used species Aspergillus nidulans which can reproduce sexually (by ascospores) and primarily asexually (by conidiospores).

A further problem in the production of heterologous proteins in filamentous fungi are the inactivation systems for heterologous DNA which especially become effective when heterologous DNA is inserted into the genome of the fungus. Gene inactivation, which was described very well, for example, for Neurospora crassa or Ascobolus immersus, can take place on different levels, i.e., on the transcriptional or on the post-transcriptional level. Similar phenomena were also described for transgenic plants. A review of this can be found in Cogoni and Macino (1999).

For Neurospora crassa the so-called RIP phenomenon was observed after DNA transformation (Singer et al., 1995; Selker, 1997), wherein multiple copies of heterologous DNA are inactivated by point mutations or DNA modifications (e.g. methylation). Consequently, foreign genes cannot be efficiently expressed in Neurospora crassa after meiotic crossing-over.

The object of the present invention is thus to provide a method for the production of heterologous protein in a filamentous fungus which allows efficient production of heterologous protein and wherein any contamination of the product protein by vegetative spores is avoided.

This object is solved according to the invention by a method for the production of heterologous protein in a filamentous fungus, comprising the cultivation of a homothallic fungus of the family Sordariaceae, which contains an expression cassette which contains the following elements in functional combination:

a promoter active in the fungus of the family Sordariaceae,

a heterologous gene and

a terminator active in the fungus of the family Sordariaceae,

and the harvesting of the protein produced in an inherently known fashion.

In the sense of the invention, a “heterologous gene” means an encoding nucleic acid sequence which originates from a different gene from the promoter contained in the expression cassette.

Unlike heterothallic species of this family or other filamentous fungi such as Aspergillus nidulans or Podospora anserina, homothallic fungi of the family Sordariaceae form neither macroconidia nor microconidia since they reproduce exclusively sexually. Arthrospores formed by decay of hyphae are also not encountered. Thus, any contamination of the product protein with vegetative spores, which presents a major problem in the production of pharmaceutically relevant proteins using filamentous fungi, is avoided.

Moreover, it has surprisingly been found that the RIP phenomenon observed after DNA transformations in Neurospora crassa and other filamentous fungi does not occur in homothallic fungi of the family Sordariaceae and especially in Sordaria macrospora. Sordaria macrospora possesses no genetic mechanisms to inactivate repetitive sequences, e.g. by methylation or point mutation. Consequently, heterologous DNA which is present in multiple copies after transformation into the genome of the fungus, is not inactivated unlike in Neurospora crassa and other filamentous fungi. No post-transcriptional gene inactivation is known in homothallic fungi of the family Sordariaceae.

The family Sordariaceae belongs to the class of Ascomycetes (sac fungi) which according to Strasburger (Lehrbuch der Botanik) is classified within the order Sphaeriales (Sordariales). As an example of the life cycle of homothallic fungi of the family Sordariaceae, the life cycle of Sordaria macrospora can be described as follows: this fungus is a haplo dikaryote which reproduces exclusively sexually. Vegetative spores, e.g. conidio spores are not formed by this fungus. The fungus is homothallic, i.e. it has a monoecious form which has no overlapping incompatibility and it can thus reproduce sexually by self-pollination (K. Esser, Cryptogamen I, 2000). The fertilization process which precedes the sexual reproduction is described as autogamous. The haploid nuclei located in the ascogon divide conjugatedly and thereby introduce the dikaryotic phase. The sexual ascospores are formed in asci (sacs). A plurality of asci (approx. 100) ripen simultaneously into fruiting bodies, the so-called perithecia. This type of fruiting body is typical for the representatives of Sordariales. After ripening the ascospores are actively ejected from the perithecia. In nature the ascospores are taken up through the food of herbivores and after gastro-intestinal passage, are released back into the open. The gastro-intestinal passage is a prerequisite for the subsequent germination of the spores on the dung of the herbivore.

The homothallic fungus of the family Sordariaceae which is used in the method according to the invention is preferably a fungus of the genus Sordaria. Especially preferred homothallic fungi of the family Sordariaceae for the implementation of the invention are Sordaria macrospora or Sordaria fimicola; other homothallic fungi of the same family which are suitable for the implementation of the method according to the invention are Neurospora linoleata, Neurospora africana, Neurospora dodgei, Neurospora galapagosensis, Neurospora pannonica and Neurospora terricola.

Sordaria macrospora, just like Sordaria fimicola is a coprophilous saphrophyte which grows on the dung of herbivores. The hyphal fungus should be classified taxonomically in the division eumycota and thus belongs to the higher fungi which have chitin walls. The complete life cycle of Sordaria macrospora is completed within seven days under laboratory conditions. Thus, the life cycle is considerably shorter than the four-week life cycle of Neurospora crassa. Molecular biological studies on the strain development of S. macrospora can thus be carried out in a considerably shorter time.

Both the formal genetic analysis and the molecular genetic analysis have been well developed for S. macrospora. In the formal genetic analyses use is made of the fact that sterile mutants of S. macrospora cannot form any self-pollination asci but that two mutants having different defects are sexually reproducible in cross-overs. That is, meiosis takes place in so-called ascogenous hyphae which results in the formation of tetrads. The result are asci with eight linearly arranged spores, of which pairs are genetically identical. The size of the spores (18×28 μm) and the linearity of the arrangement allow technically simple formal genetics on account of the ordered tetrads. In addition, a plurality of mutants have been described in the meantime, relating to both the physiological and the morphological properties of the fungus (Esser and Straub, 1958; Nowrousian et al., 1999; Masloff et al., 1999; Le Chevanton and Leblon, 1989). An indexed cosmid gene bank which can be used to isolate genes of S. macrospora was described by Pöggeler et al. (1997).

The codon usage in the host organism is decisive for an optimal expression of heterologous genes. From the listing of the most common amino acid codons within protein coding genes in Table 1 it can be seen that there is an astonishing agreement between the most commonly used amino acid codons in Sordaria macrospora, Drosophila melanogaster and primates. In contrast to this, clear differences can be identified for E. coli or for Saccharomyces cerevisiae. As a result of the similarity of the codon usage, S. macrospora is thus an excellent host system for the production of heterologous proteins of human origin.

Homothallic fungi of the family Sordariaceae can be grown simply and cheaply in laboratory cultures. As eukaryotic microorganisms they are also capable of carrying out post-translational modification of recombinant eukaryotic proteins. Another factor in favor of the biotechnical use of homothallic fungi of the family Sordariaceae is that no human-, animal- or plant-pathogenic organisms are involved. Moreover, recombinant strains of Sordaria macrospora can also be produced by genetic engineering methods in combination with classical (conventional) cross-overs, which is a considerable advantage over imperfect fungi of the genus Aspergillus.

The only spores which are formed by homothallic fungi of the family Sordariaceae and especially by S. macrospora and S. fimicola are so-called ascospores which appear in the fruiting body and perithecia in the course of sexual reproduction. Such ascospores are very much larger, heavier and formed in significantly greater numbers than the conidiospores. They thus possess a very low dissemination capability. No ascospores are generally formed in submerse cultures or in shake cultures. If the heterologous protein produced is to be absolutely spore-free, in an especially preferred embodiment of the method for the production of heterologous protein according to the invention, a sterile mutant of the homothallic fungus of the family Sordariaceae is used. Sterile mutant strains which can be obtained, for example, by mutagenesis of protoplasts with EMS or by irradiation with UV light, have no reproductive structures and thus produce no ascospores. For example, in the method for the production of heterologous protein according to the invention, sterile mutants of S. macrospora (Esser and Straub, 1958; Masloff et al., 1999; Nowrousian et al., 1999) can be used.

Cultivation of the homothallic fungus used for the production of heterologous protein preferably takes place at a temperature of 27±2° C. This growth optimum is significantly lower than that for Neurospora crassa which lies above 30° C. As an important safety-relevant aspect it should be noted that S. macrospora dies off at temperatures above 32° C.

According to a preferred embodiment of the invention, the promoter active in the fungus of the family Sordariaceae, under whose control the heterologous gene is expressed, originates from a filamentous fungus. This promoter can, for example, be the gpd promoter from Aspergillus nidulans, but the promoter is preferably a promoter from Sordaria macrospora. Especially preferred promoters are the cpc2 promoter, the ndk1 promoter, the acl1 promoter or the ppg1 promoter from Sordaria macrospora. The cpc2 promoter and the ndk1 promoter from S. macrospora are described below. The acl1 gene of S. macrospora was described by Nowrousian et al (1999). The ppg1 gene of S. macrospora was described by Pöggeler (2000).

The terminator active in the fungus of the family Sordariaceae preferably originates from a filamentous fungus. The terminator can, for example, be the trpC terminator from Aspergillus nidulans (Mullaney et al., 1985) or a terminator from Sordaria macrospora. Especially preferred terminators from S. macrospora are the cpc2 and ndk1 terminators described here, the acl1 terminator (Nowrousian et al., 1999) and the ppg1 terminator (Pöggeler, 2000).

The heterologous gene preferably encodes a protein glycosylated after expression in eukaryonts. The heterologous gene can, for example, be a growth factor, a cytokine, a clotting factor, an industrial protein or a technical enzyme. Especially preferably the heterologous gene encodes one of the following proteins: G-CSF, GM-CSF, IL-1, IL-2, IL-4, IL-6, IL1ra, IFN-α, IFN-β, IFN-γ, erythropoietin, glucoamylase, clotting factor VIII, clotting factor XII, clotting factor XIII, human serum albumin.

In a further embodiment of the method according to the invention between the promoter and the heterologous gene in the reading frame with the heterologous gene there is arranged a sequence which encodes a signal sequence which functions in the fungus of the family Sordariaceae. The signal sequence is preferably a signal sequence from a filamentous fungus, for example, the signal sequence of the glucoamylase from Aspergillus niger (Gordon et al. 2000; Gouka et al. 1997). Signal sequences from Sordaria macrospora, e.g. the signal sequence of the ppg1 gene from S. macrospora are especially preferred.

A further object of the invention is to provide promoters which can be used in the method according to the invention for the production of heterologous protein.

This object is solved by a nucleic acid molecule, comprising:

(1) a promoter active in a homothallic fungus of the family Sordariaceae, which is selected from the following nucleic acids:

(a) a nucleic acid having the sequence specified in SEQ ID NO:1;

(b) a nucleic acid having the sequence specified in SEQ ID NO:2;

(c) a nucleic acid having a sequence which exhibits at least 50% identity with one of the sequences specified in (a) or (b);

(d) a nucleic acid which hybridizes with the opposite strand of a nucleic acid specified in (a) or (b);

(e) a derivative obtained by substitution, addition and/or deletion of one or a plurality of nucleotides of a nucleic acid specified in (a) or (b);

(f) a fragment of one of the nucleic acids specified in (a) to (e) which retains the function of the promoter active in the fungus of the family Sordariaceae;

(g) a combination of a plurality of nucleic acids specified in (a) to (f) wherein the sequences of the nucleic acids can be the same or different; or

(2) a nucleic acid having a sequence which is complementary to the sequence of one of the nucleic acids specified in (a) to (g).

In the sense of the invention the expression “% identity” refers to identity on the DNA level which can be determined by known methods, e.g., computer-aided sequence comparisons (Altschul et al., 1990).

The expression “identity” known to the person skilled in the art describes the degree of relationship between two or more DNA molecules, which is determined by the matching between the sequences. The percentage of the “identity” is obtained from the percentage of identical regions in two or more sequences taking into account gaps or other sequence characteristics.

The identity of interrelated DNA molecules can be determined using known methods. Special computer programs with algorithms taking account of the particular requirements are usually used. Preferred methods for the determination of identity initially generate the greatest matching between the sequences studied. Computer programs for the determination of identity between two sequences comprise, but are not restricted to the GCG program package, including GAP (Devereux et al., 1984; Genetics Computer Group University of Wisconsin, Madison, (Wisc.)); BLASTP, BLASTN and FASTA (Altschul et al., 1990). The BLAST X program can be obtained from the National Center for Biotechnology Information (NCBI) and from other sources (BLAST Handbook, Altschul S. et al., NCB NLM NIH Bethesda Md. 20894; Altschul et al., 1990). The known Smith Waterman algorithm can also be used to determine identity.

Preferred parameters for the sequence comparison comprise the following:

Algorithm: Needleman and Wunsch, J. Mol. Biol. 48: 443-453 (1970)

Comparison matrix: Matches=+10,

Mismatch=0

Gap penalty: 15

Gap length penalty: 1

The GAP program is suitable for use with the preceding parameters. The preceding parameters are the default parameters for nucleic acid sequence comparisons.

Other exemplary algorithms, gap opening penalties, gap extension penalties, comparison matrices, including those specified in the Program Handbook, Wisconsin Package, Version 9, September 1997, can be used. The choice will depend on the comparison to be made and furthermore on whether the comparison is made between sequence pairs, where GAP or Best Fit are preferred, or between a sequence and a comprehensive sequence database where FASTA or BLAST are preferred.

The feature “sequence which hybridizes with the opposite strand of a sequence from (a) or (b)” indicates a sequence which hybridizes under stringent conditions with a sequence having the features specified under (a) or (b). For example, the hybridizations can be carried out at 68° C. in 2×SSC. Examples of stringent conditions are given in Sambrook et. al. (1989).

The sequences specified in SEQ ID NO:1 or SEQ ID NO:2 correspond to the promoter sequence of the ndk1 or cpc2 gene isolated from S. macrospora. The nucleic acid sequence specified in (c) can also exhibit at least 60%, 70%, 80% or 90% identity with the sequences specified in (a) or (b). Especially preferably it exhibits 95% identity with one of these sequences.

Moreover, the invention also provides a vector for the transformation of a homothallic fungus of the family Sordariaceae which contains the following elements in functional combination:

a promoter active in the fungus of the family Sordariaceae,

a heterologous gene,

a terminator active in the fungus of the family Sordariaceae and

a selection marker.

The promoter active in the fungus of the family Sordariaceae of the vector according to the invention can be the gpd promoter from Aspergillus nidulans. However, the nucleic acids described previously under (1) are especially preferred as promoters. The acl1 and the ppg1 promoter from Sordaria macrospora are likewise preferred.

The terminator contained in the vector according to the invention can be the trpc terminator from Aspergillus nidulans but the cpc2 terminator, the ndk1 terminator, the acl1 terminator or the ppg1 terminator from Sordaria macrospora are especially preferred. Suitable selection markers for example are the ura5 gene from Sordaria macrospora (Le Chevanton and Leblon, 1989) or Podospora anserina (Turcq and Begueret, 1987). A hygromycin B-resistance gene (see, for example, Kaster et al., 1983) is preferably used as a selection marker.

The invention further provides a host organism. This host organism is a homothallic fungus of the family Sordariaceae which contains a vector according to the invention. The host organism preferably belongs to the genus Sordaria, the host organism is especially preferably Sordaria macrospora or Sordaria fimicola. In an especially preferred embodiment the host organism is a sterile strain which forms neither asexual mitospores nor sexual meiospores.

The invention further provides a kit comprising:

(a) a vector according to the invention and

(b) a homothallic fungus of the family Sordariaceae suitable for the production of heterologous protein.

The nucleic acid molecule, the vector, the host organism and the kit according to the invention can be used for expression of a heterologous gene under the control of the promoter or for the manufacture of one or a plurality of proteins.

The following figures and examples explain the invention.

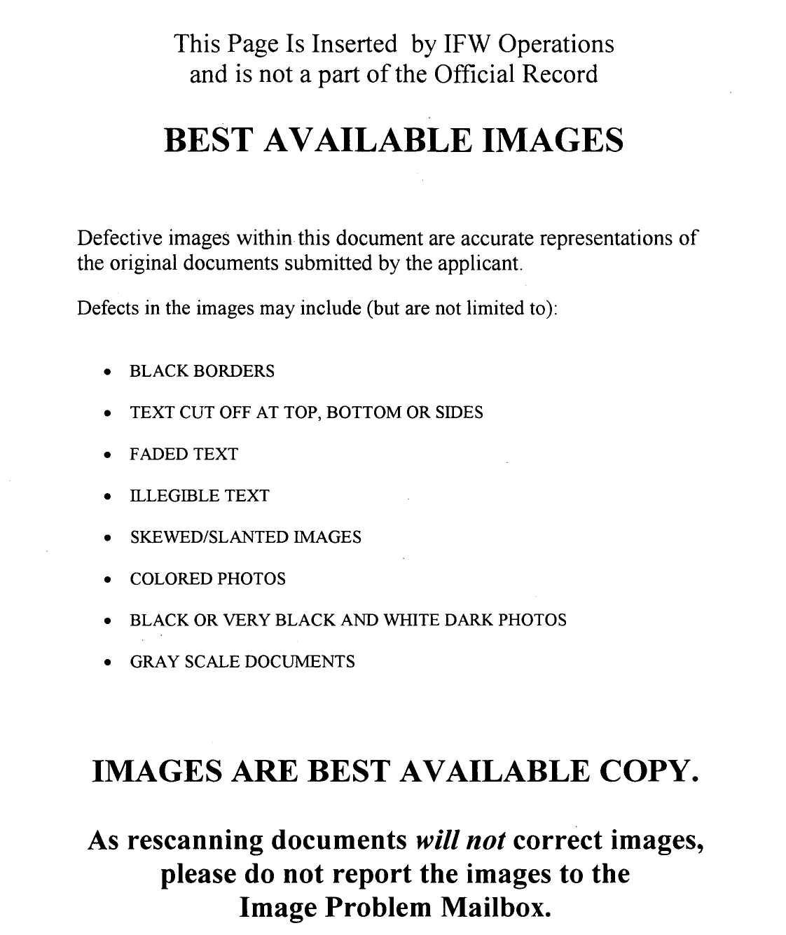

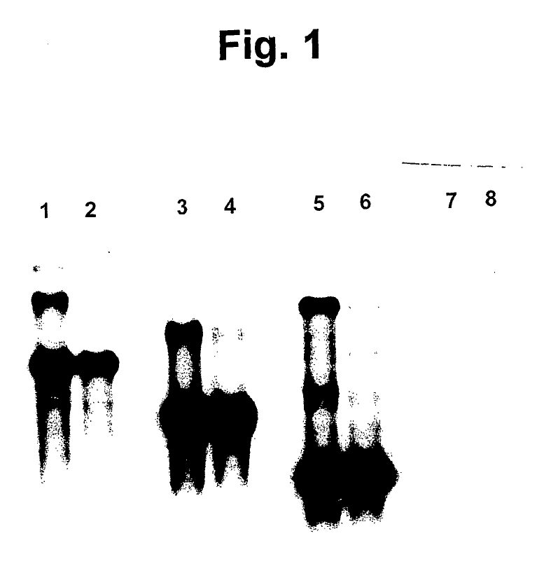

FIG. 1 shows an autoradiogram of a Northern Hybridization. 5 μm mRNA of S. macrospora was applied in the individual traces, from the wild type strain in the odd traces, from the sterile mutant inf in the even traces. The acl1 gene (trace 1, 2), the cpc2 gene (trace 3, 4), the ndk1 gene (trace 5, 6) and the ppg1 gene (trace 7, 8) were used as probes. A 2.7 kb acl1 transcript can be identified in traces 1, 2, a 1.7 kb cpc2 transcript in traces 3, 4, a 1.5 kb ndk1 transcript in traces 5, 6 and a 0.65 kb ppg1 transcript in traces 7, 8.

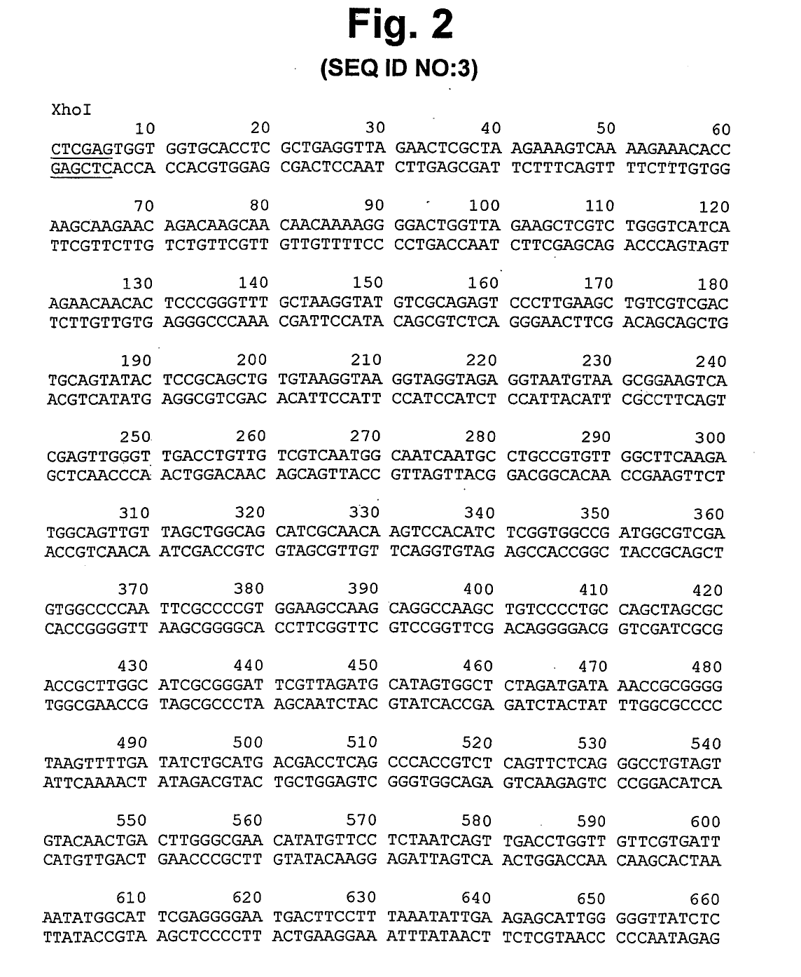

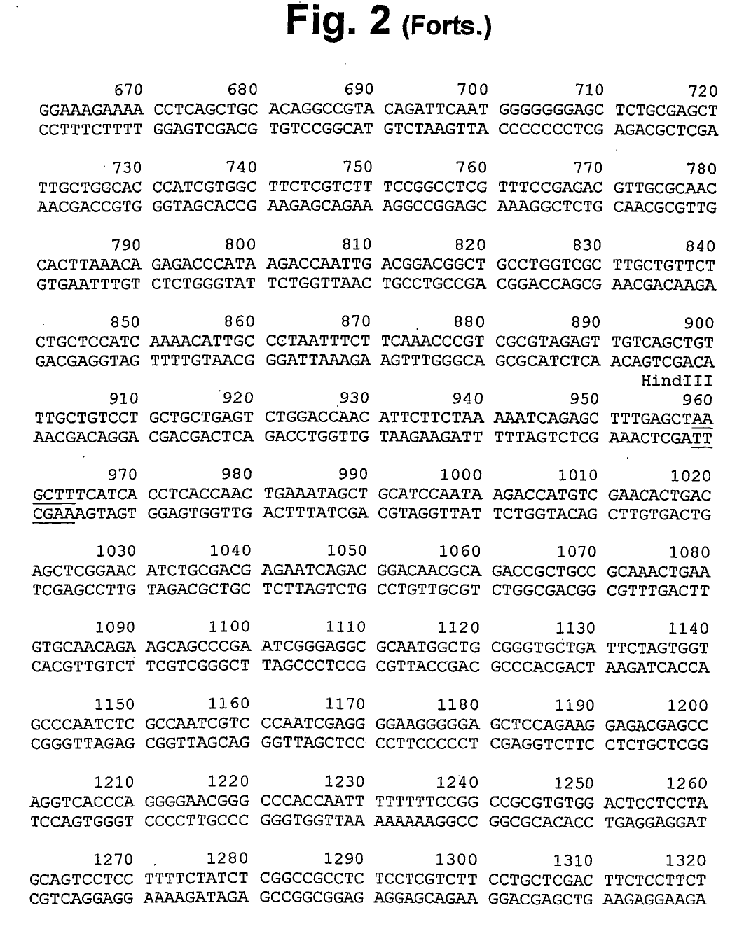

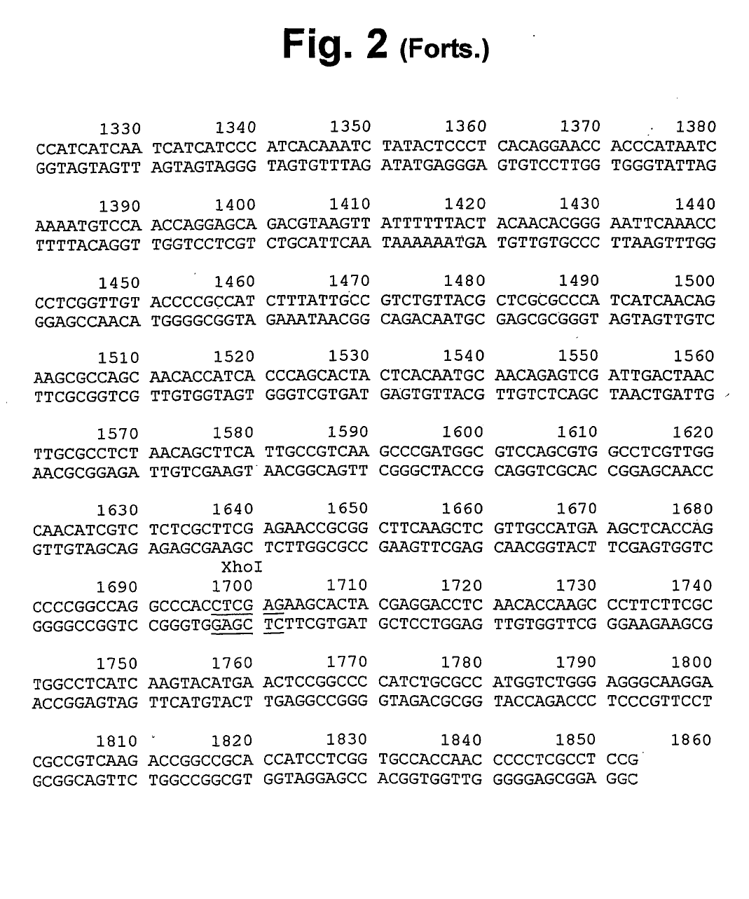

FIG. 2 shows the nucleotide sequence of the ndk1 gene (part fragment) of S. macrospora including the approx. 1.4 kb promoter region.

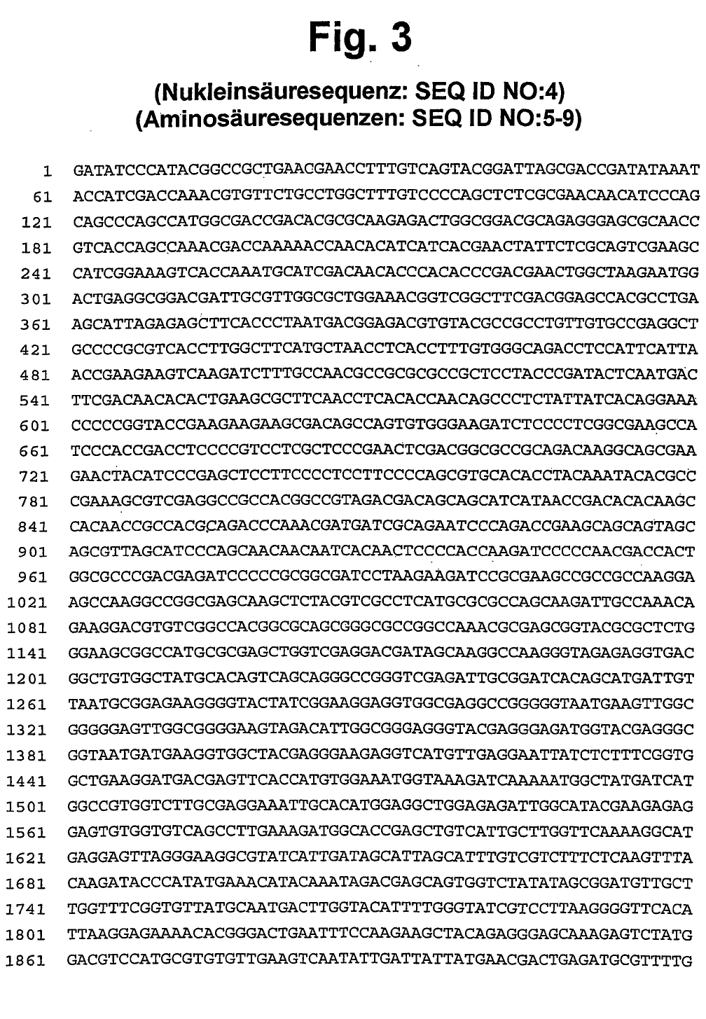

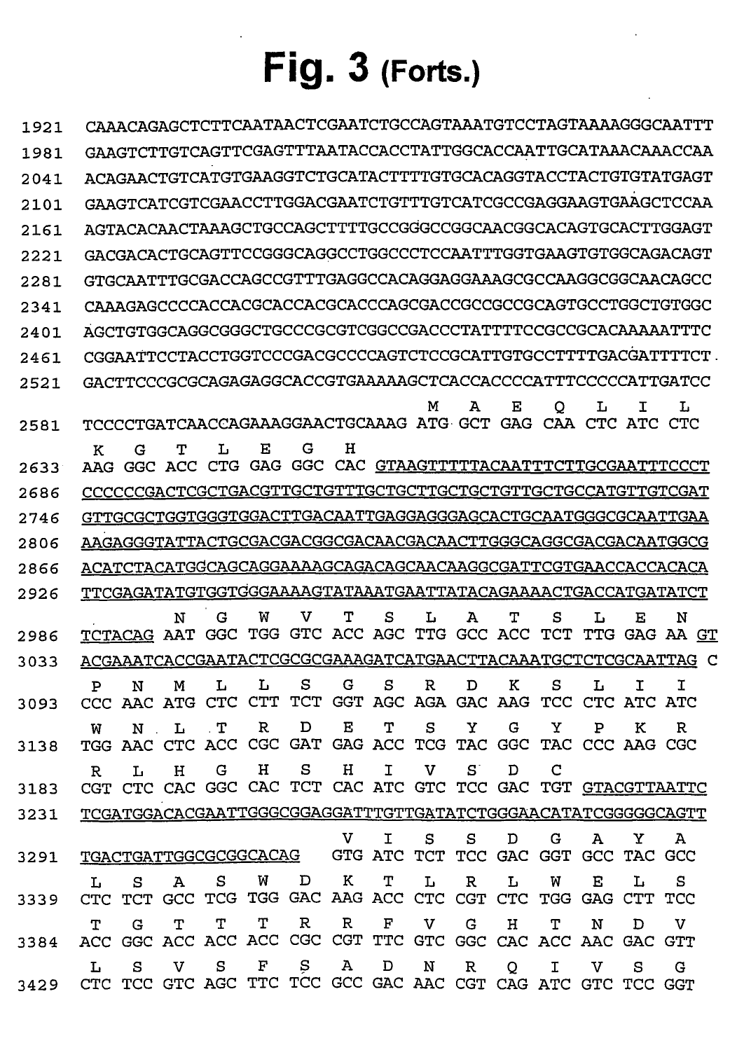

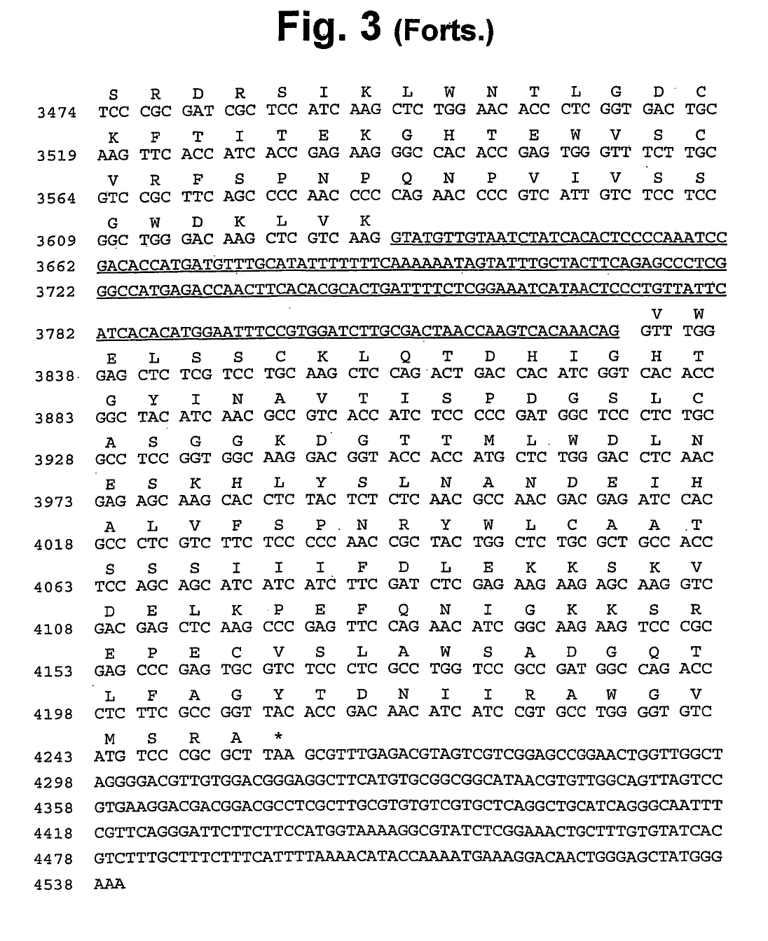

FIG. 3 shows the nucleotide sequence of the cpc2 gene of Sordaria macrospora and the amino acid sequences derived therefrom. The intron sequences (4) are characterized by underlining, the promoter is located in the section of nucleotide 1-2611, the termination sequence can be found in the region between nucleotide 4258 and nucleotide 4539.

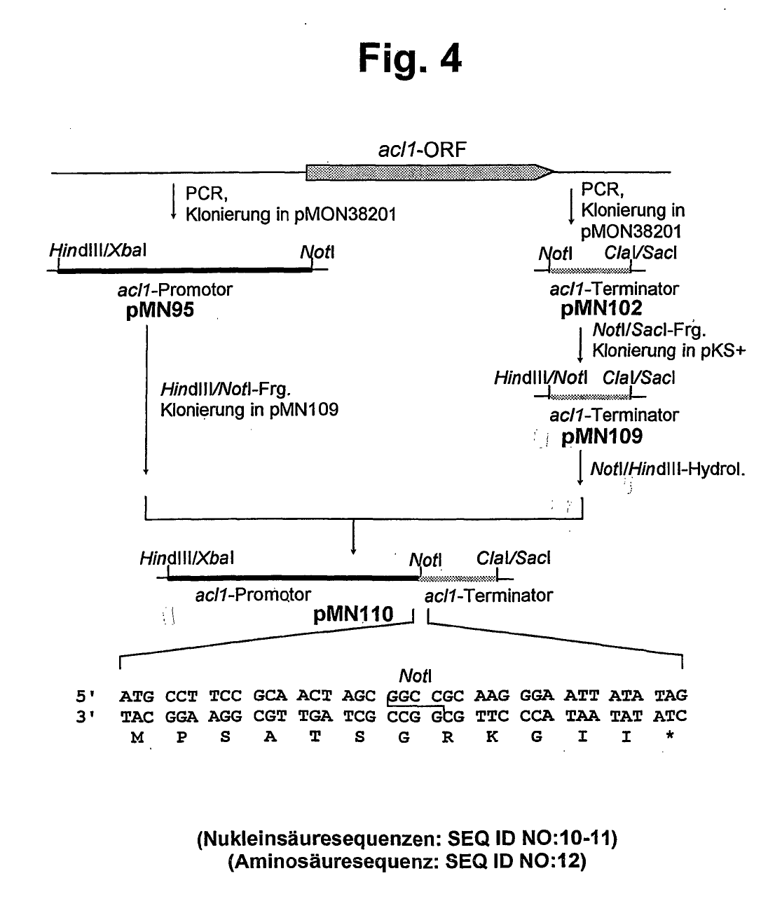

FIG. 4 shows the cloning scheme for the pMN110 expression vector.

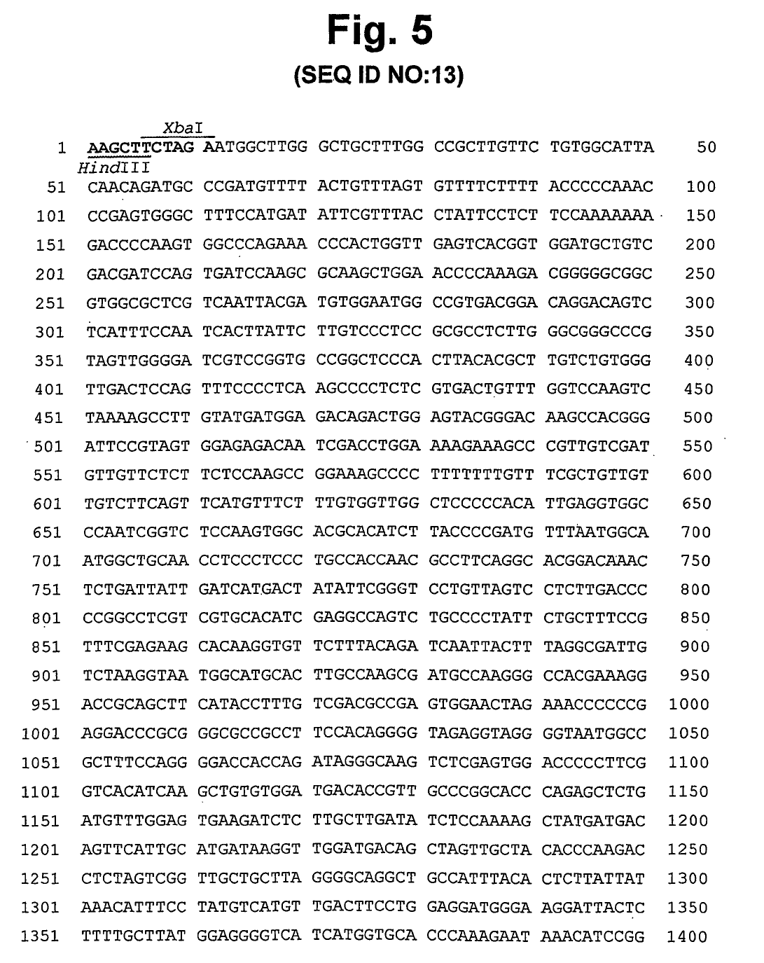

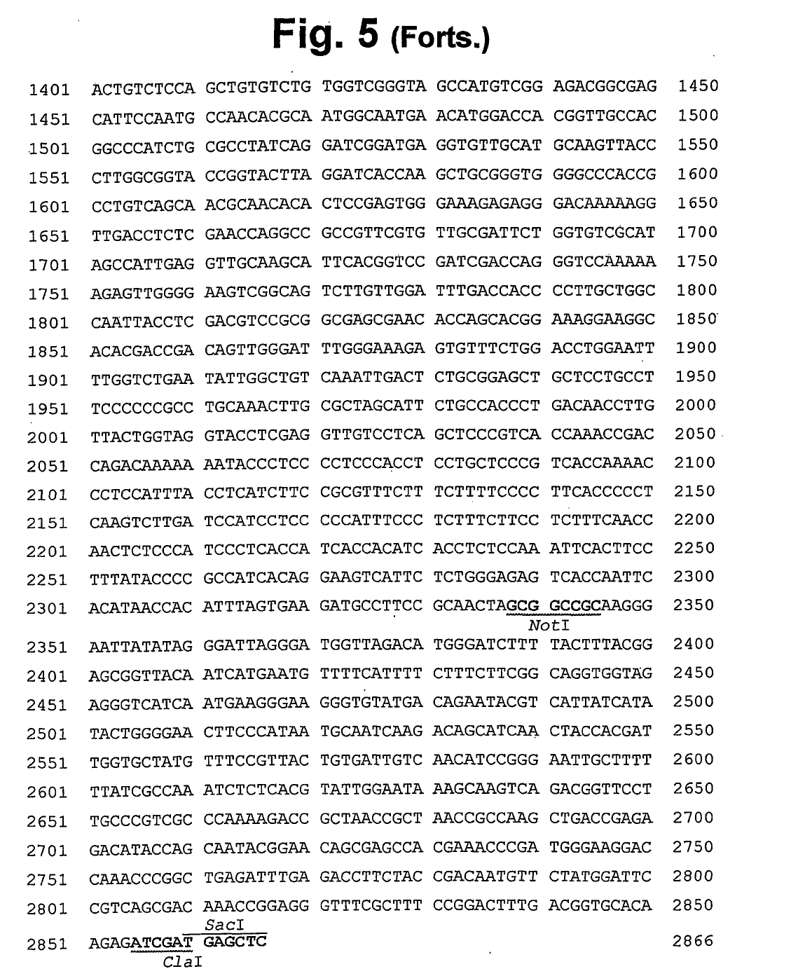

FIG. 5 shows the nucleotide sequence (promoter and terminator) of the pMN110 plasmid. The sequences originate from the acd1 gene of S. macrospora.

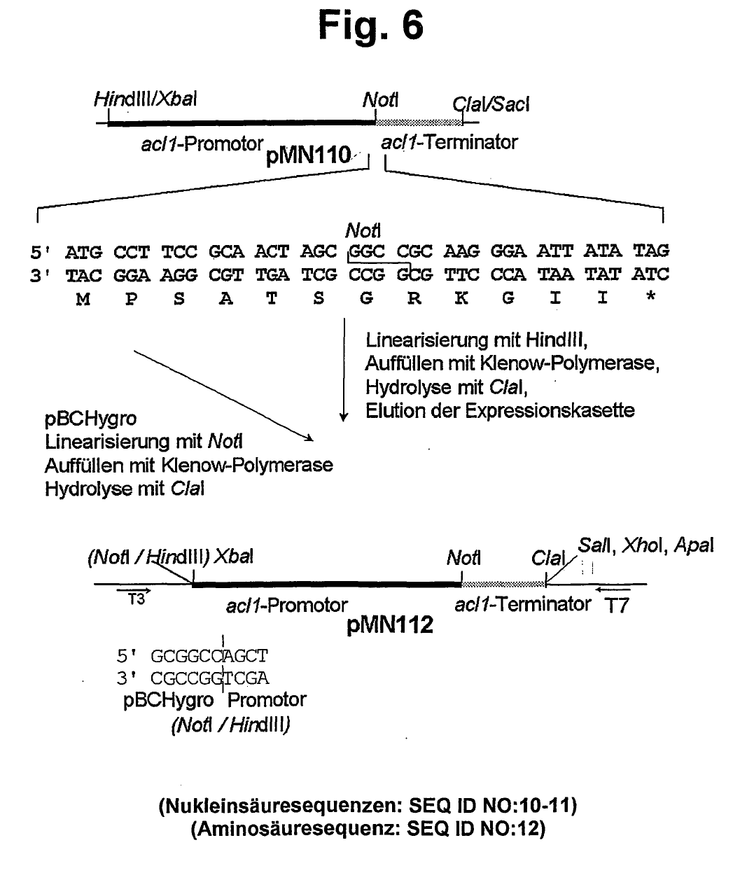

FIG. 6 shows the cloning scheme for the pMN112 expression vector.

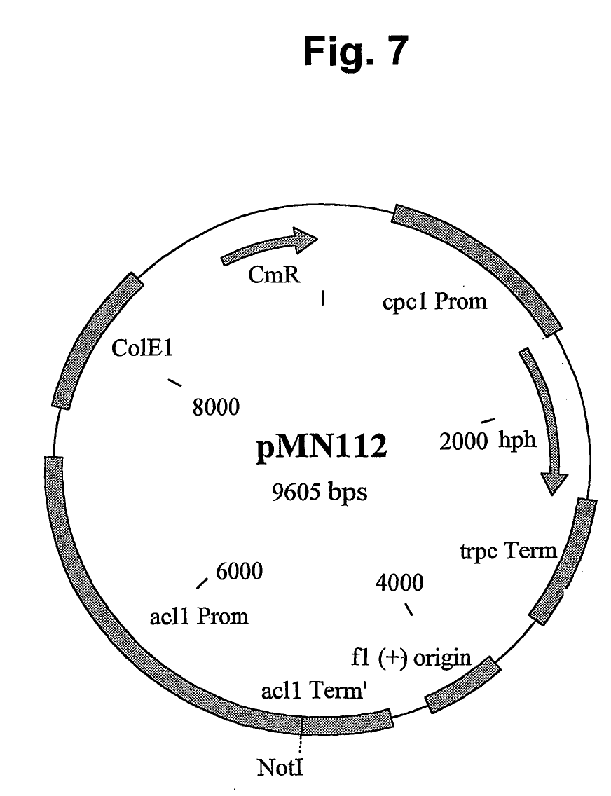

FIG. 7 shows the physical-genetic map of the pMN112 plasmid.

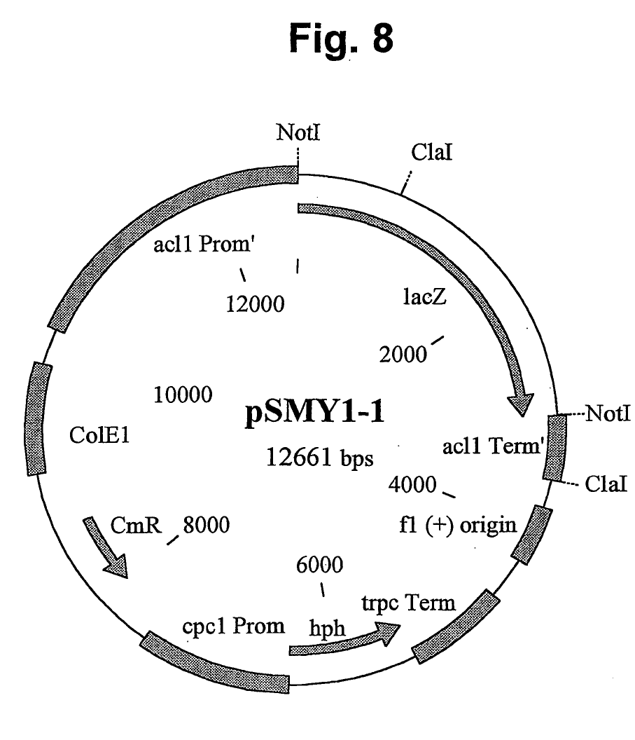

FIG. 8 shows the physical-genetic map of the pSMY1-1 plasmid.

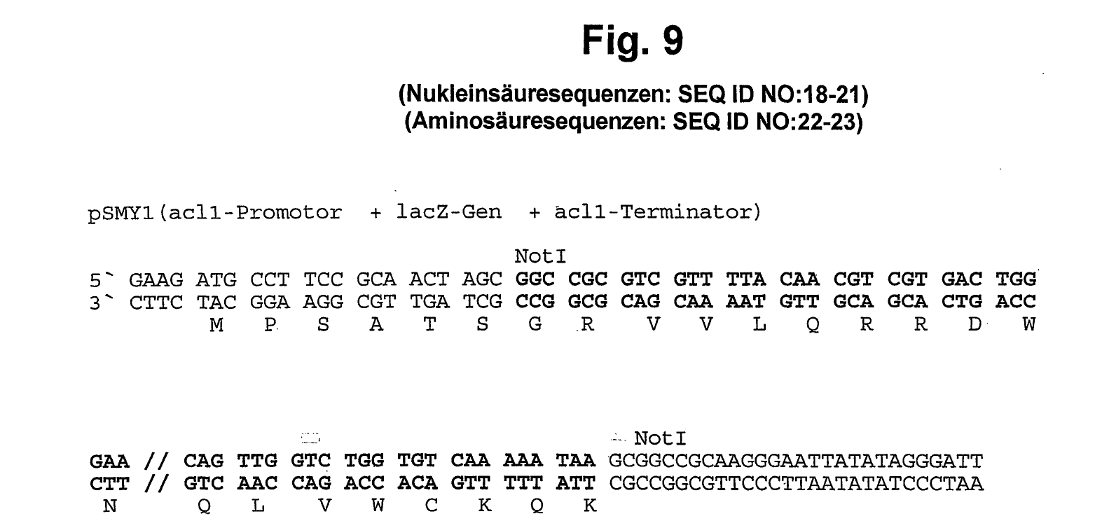

FIG. 9 shows the nucleotide sequence in the vicinity of the ATG start codon of the lacZ gene of the pSMY1 plasmid.

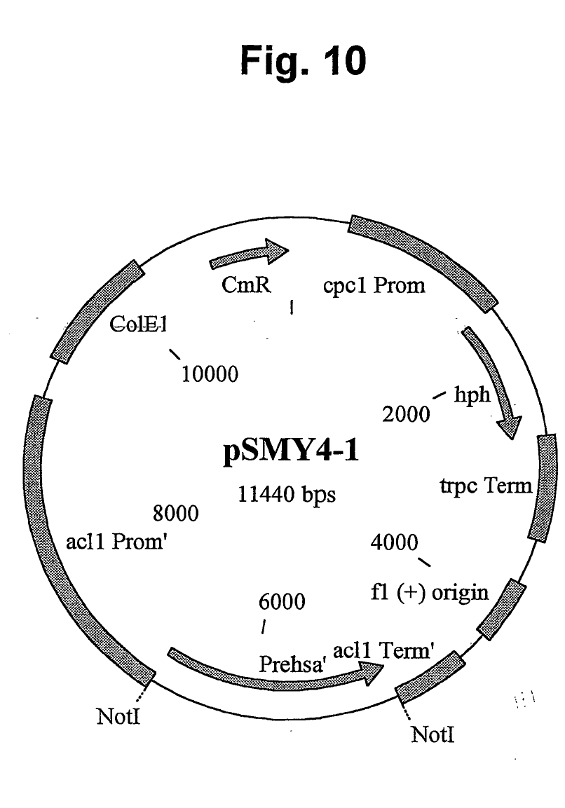

FIG. 10 shows the physical-genetic map of the pSMY4-1 plasmid.

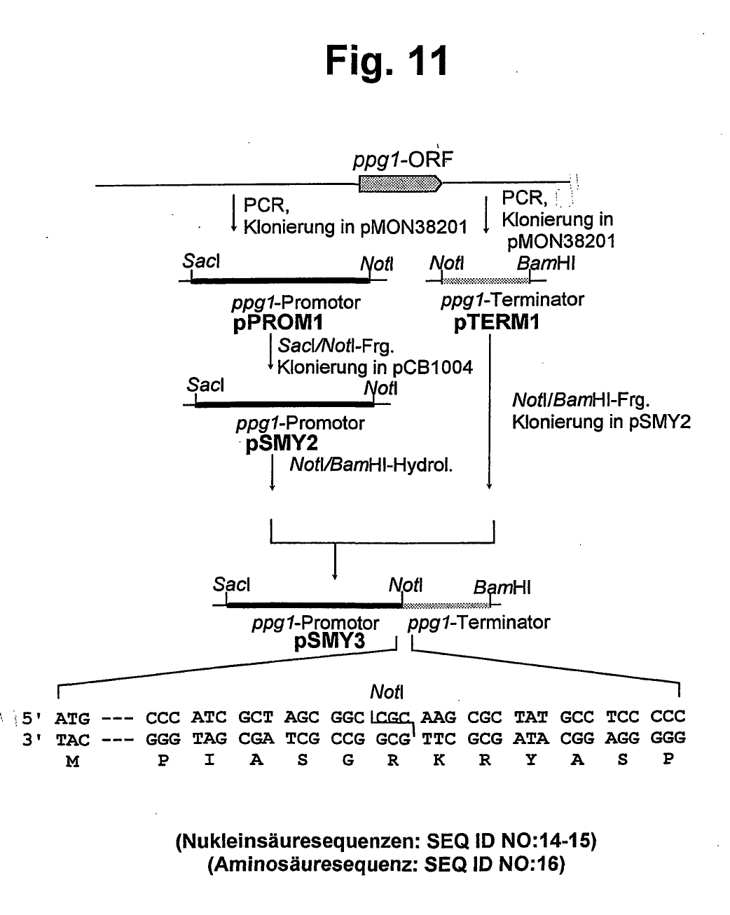

FIG. 11 shows the cloning scheme for the pSMY3 expression vector.

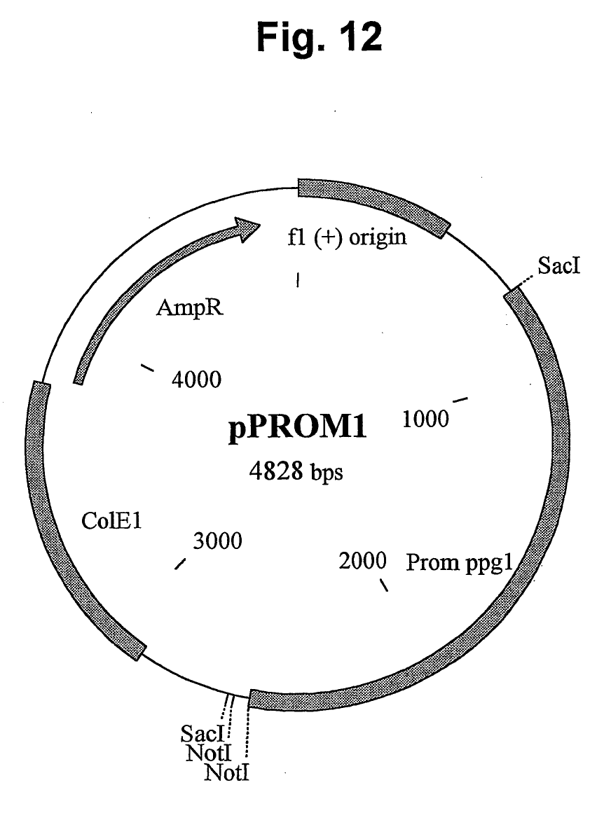

FIG. 12 shows the physical-genetic map of the pPROM1 plasmid.

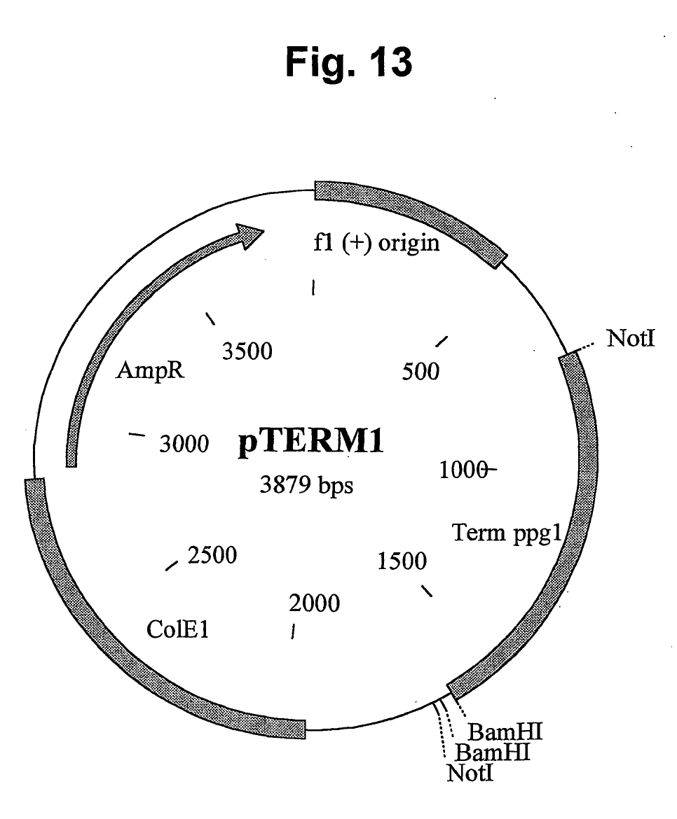

FIG. 13 shows the physical-genetic map of the pTERM1 plasmid.

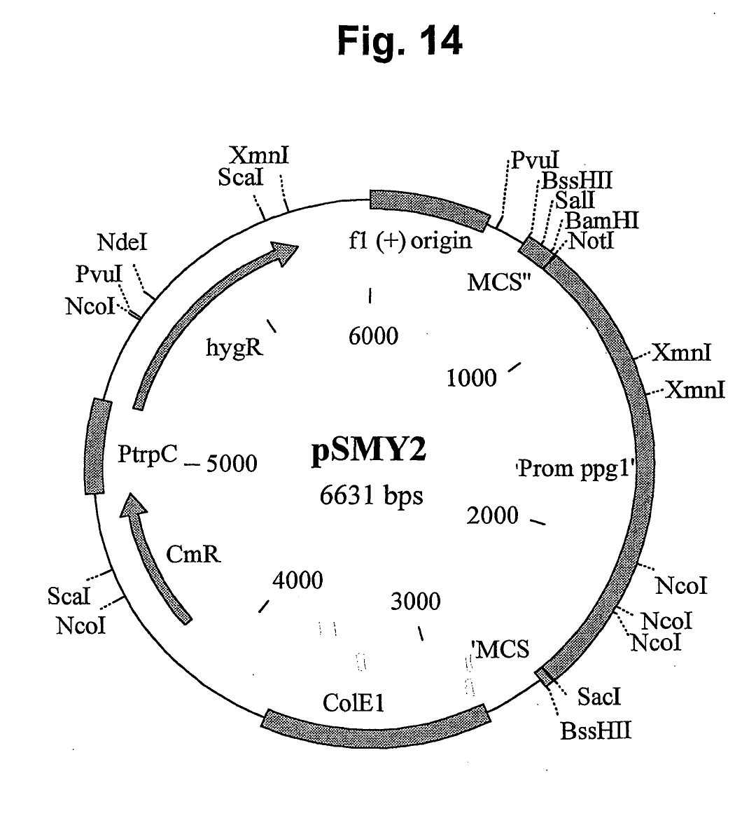

FIG. 14 shows the physical-genetic map of the pSMY2 plasmid.

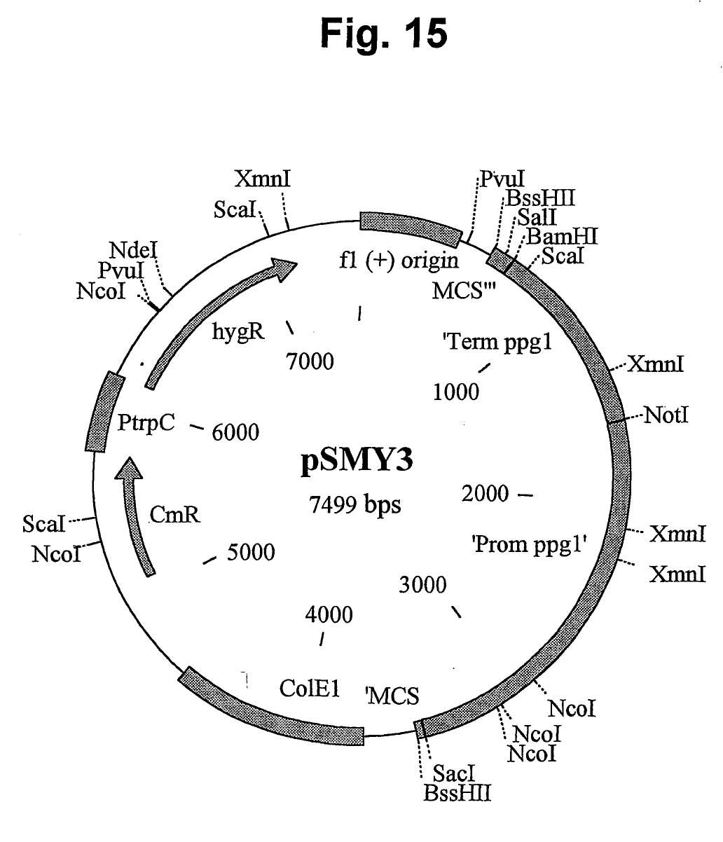

FIG. 15 shows the physical-genetic map of the pSMY3 plasmid.

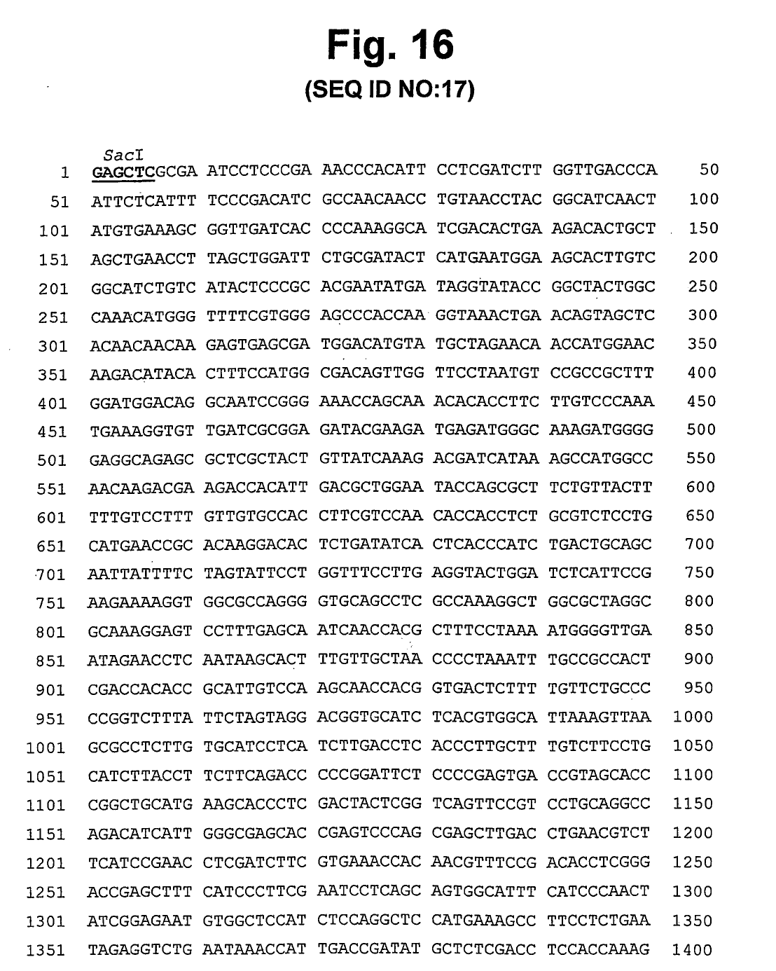

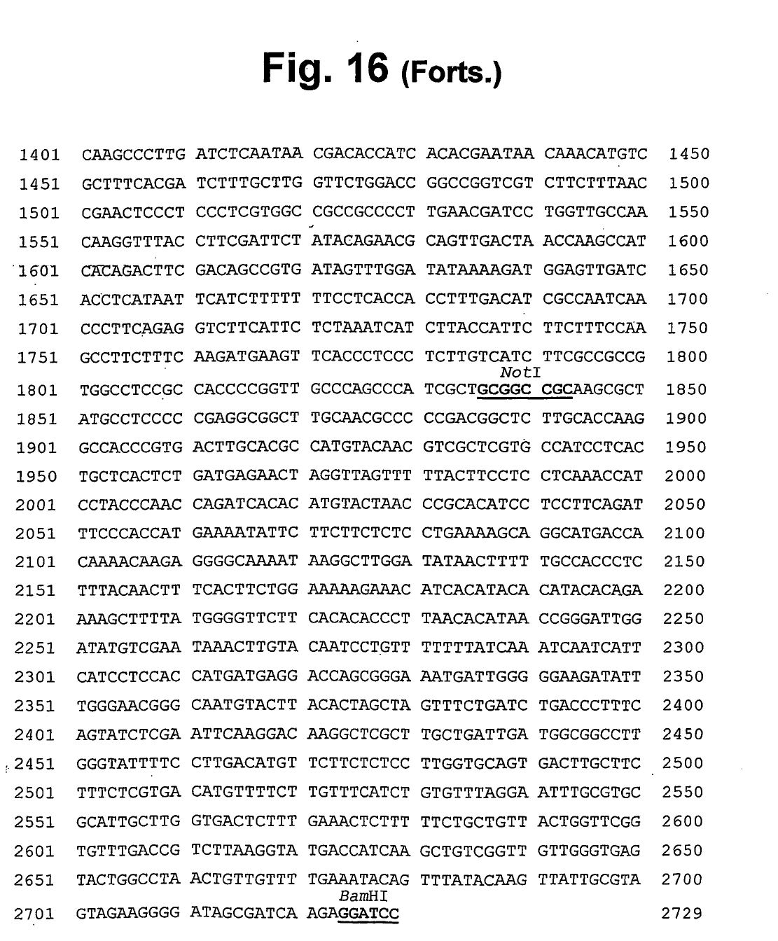

FIG. 16 shows the nucleotide sequence of the insert fragment of the pSMY3 plasmid.

FIG. 17 shows the physical-genetic map of the pSE40-6 plasmid.

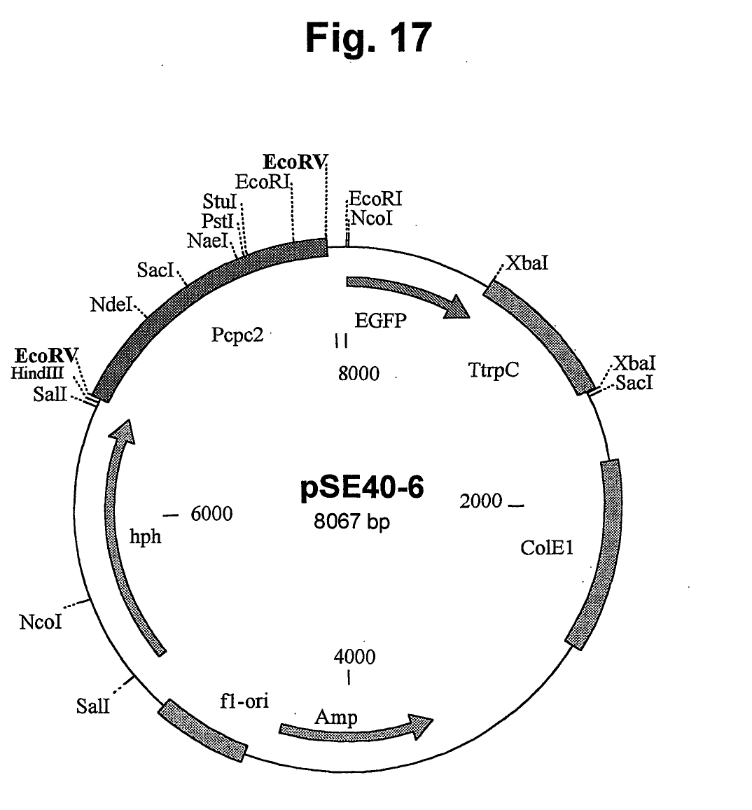

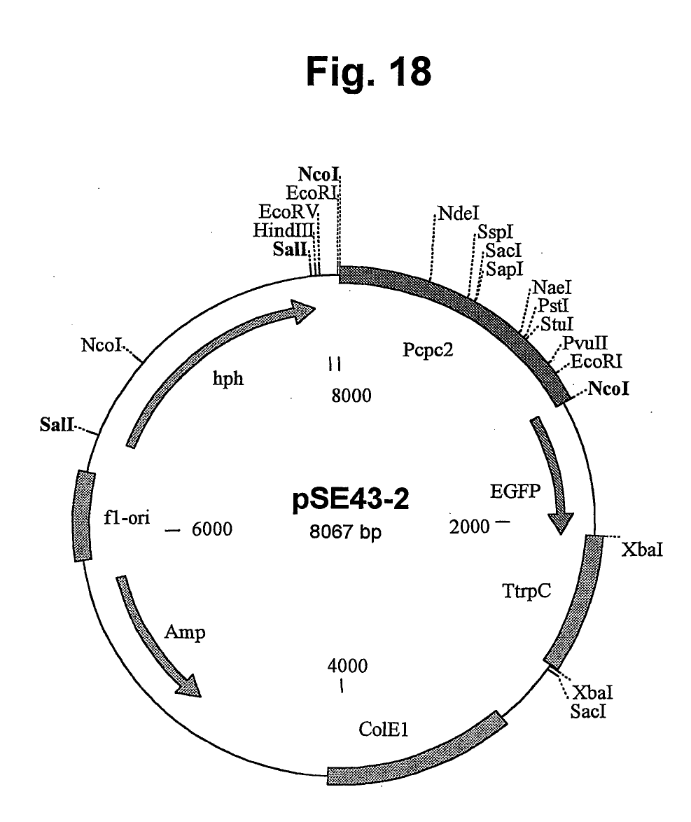

FIG. 18 shows the physical-genetic map of the pSE43-2 plasmid.

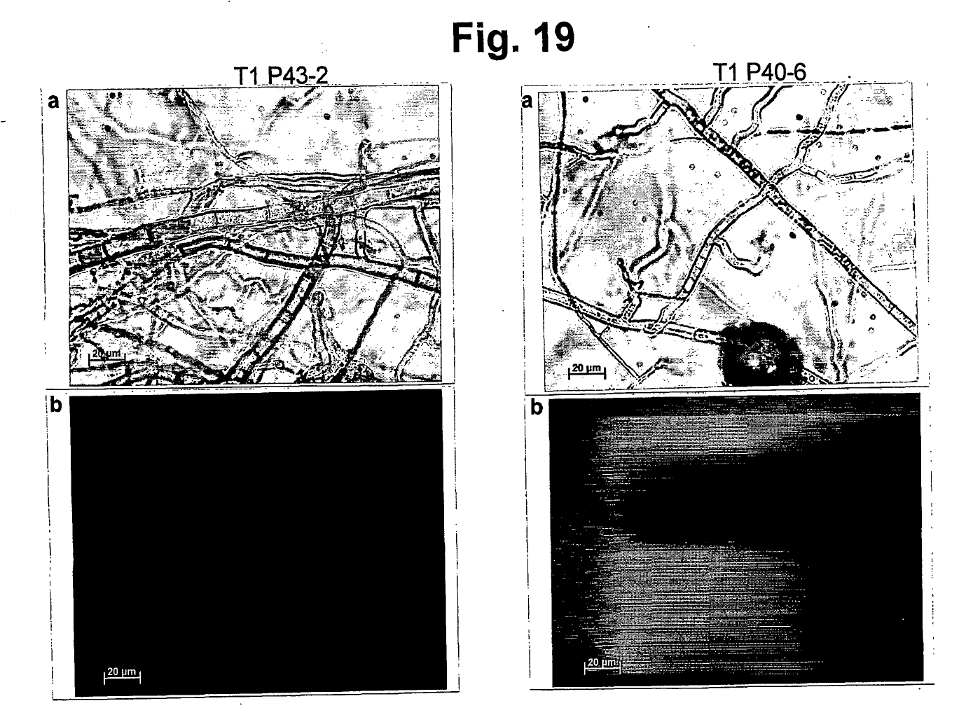

FIG. 19 shows micrographs of transgenic S. macrospora strains (T1p40-6, T1p43-2) which carry the plasmids pSE40-6 or pSE43-2. (a) Interference micrograph and (b) fluorescence micrograph.

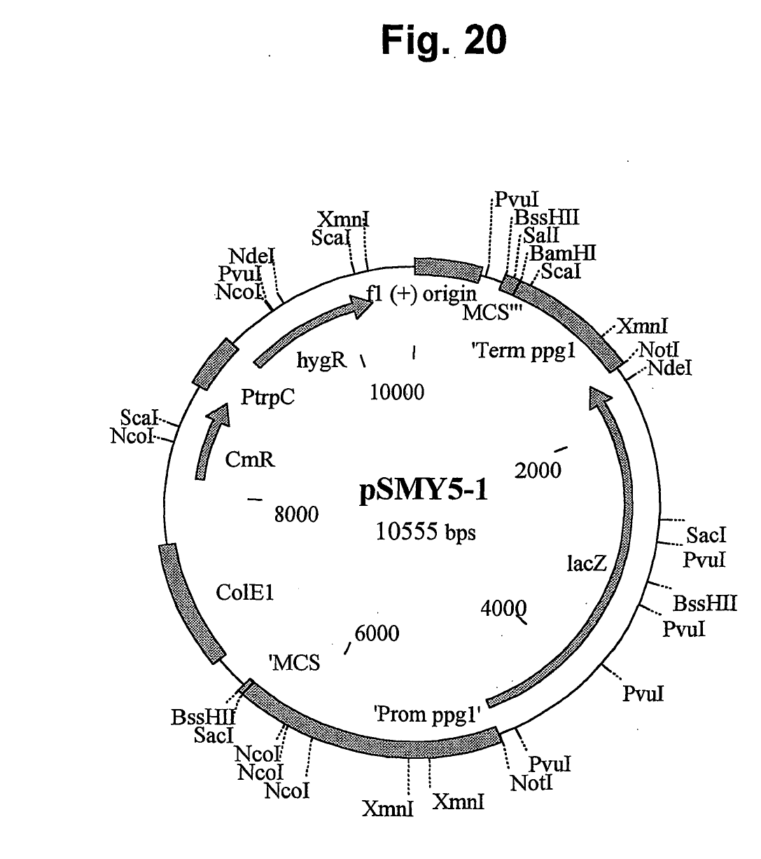

FIG. 20 shows the physical-genetic map of the pSMY5-1 plasmid.

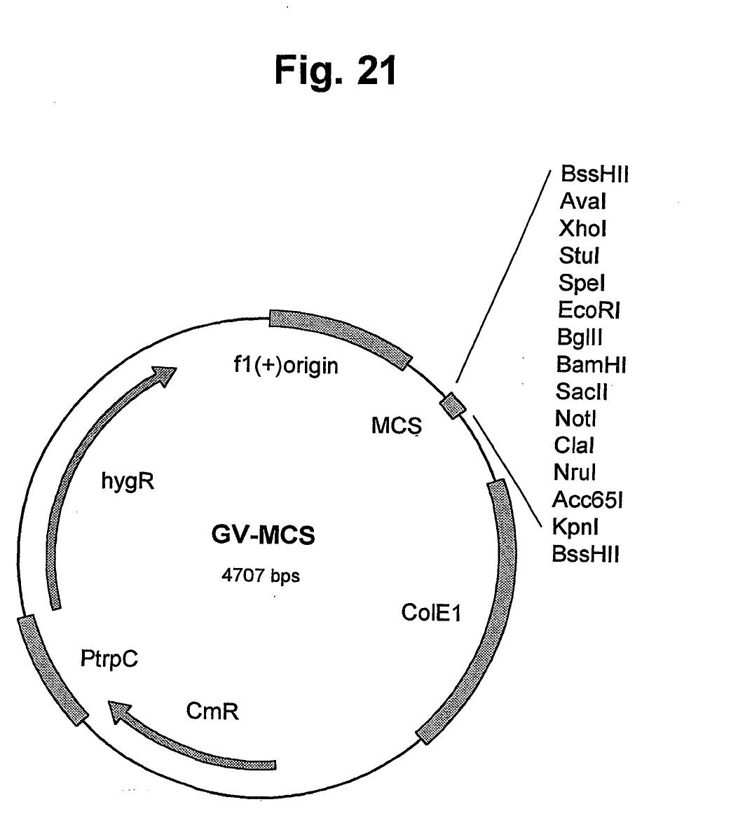

FIG. 21 shows the physical-genetic map of the GV-MCS plasmid.

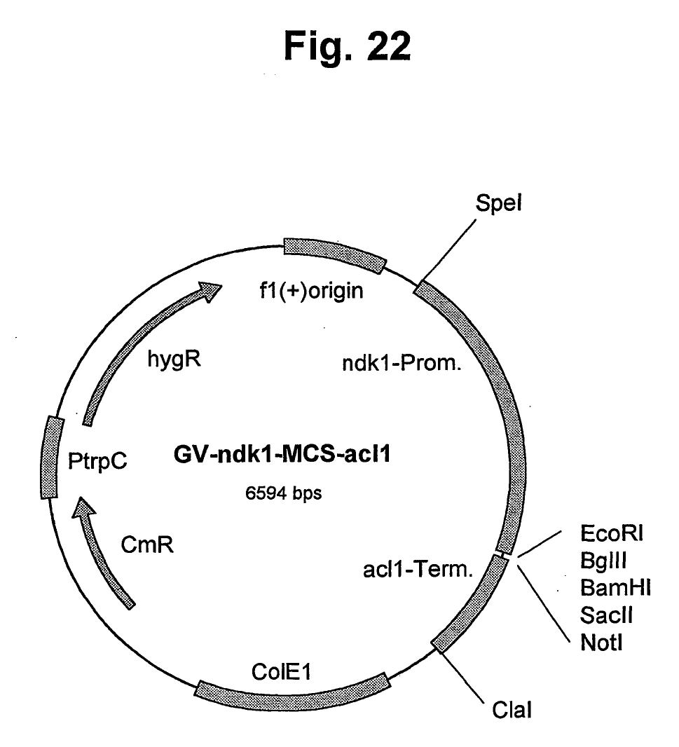

FIG. 22 shows the physical-genetic map of the GV-ndk1-MCS-acl1 plasmid.

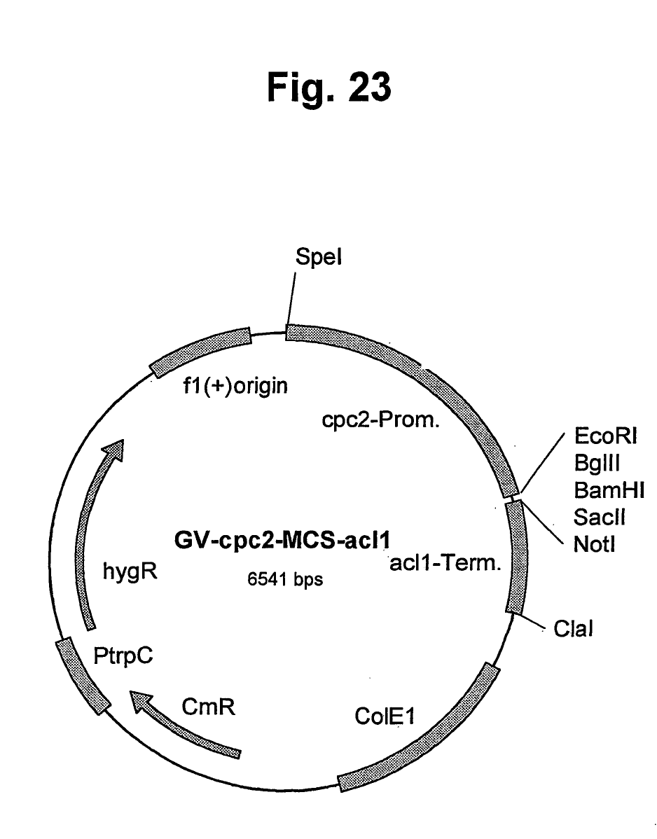

FIG. 23 shows the physical-genetic map of the GC-cpc2MCS-acl1 plasmid.

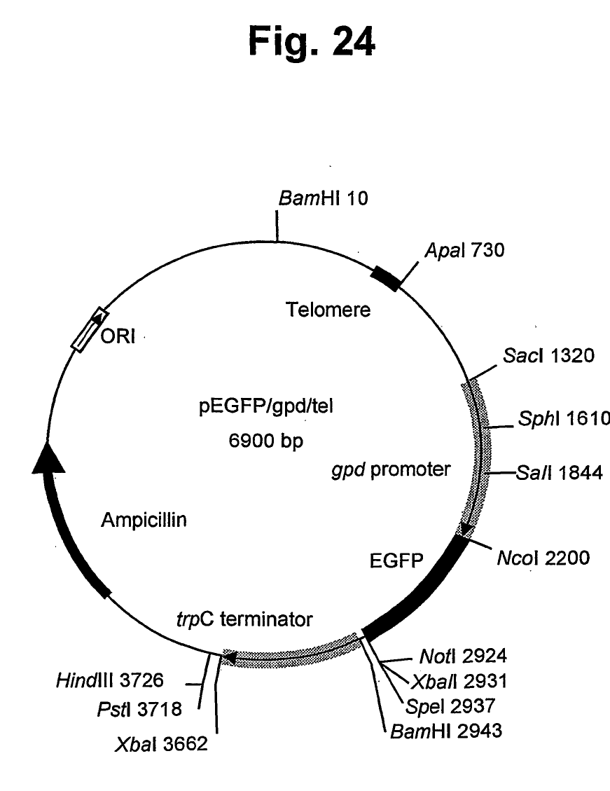

FIG. 24 shows the plasmid pEGFP/gpd/tel which was used as the starting plasmid for cloning the plasmids pSM1 and pSM2.

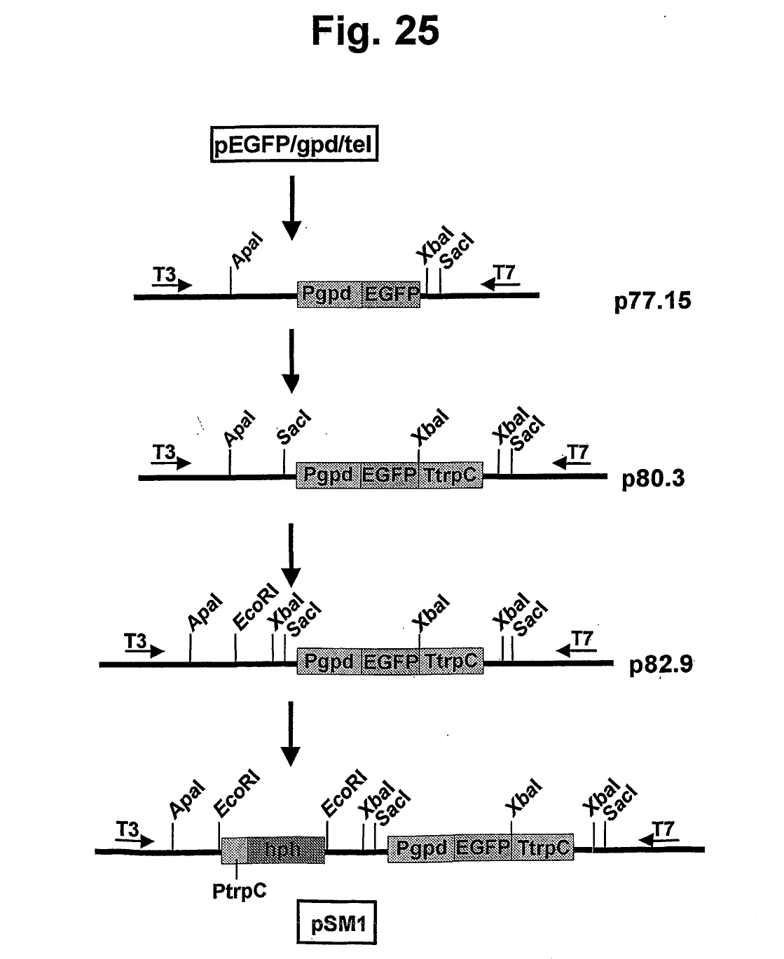

FIG. 25 shows the cloning scheme of the pSM1 plasmid.

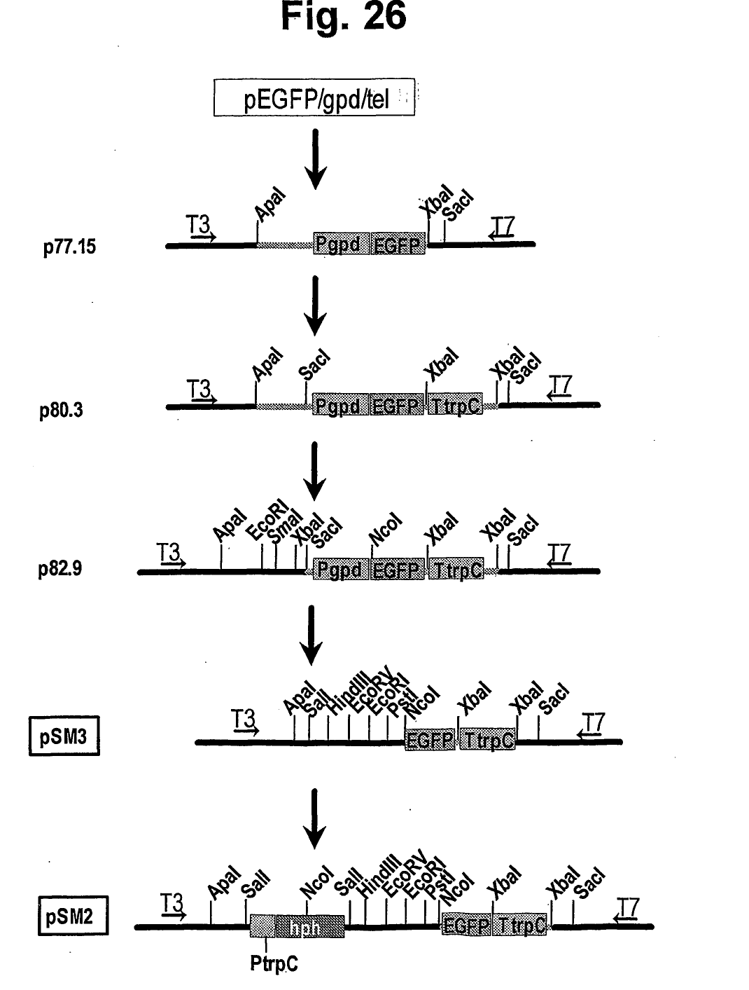

FIG. 26 shows the cloning scheme of the pSM2 plasmid.

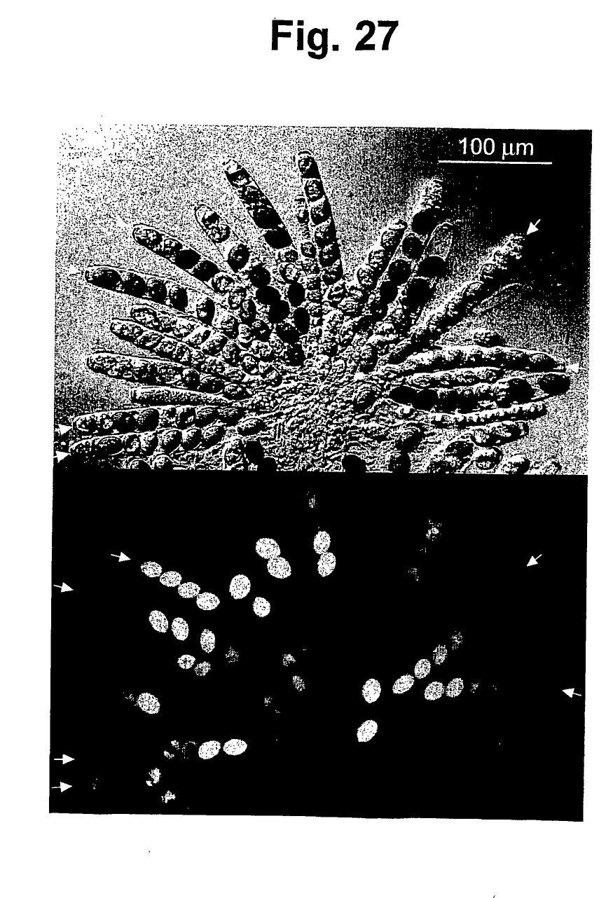

FIG. 27 shows the fluorescence microscopic analysis of asci with ascospores from S. macrospora transformands which carry plasmid pEGFP/gpd/tel. The imaged strain originates from a crossover of the S. macrospora transformand T-EG2 and the color spore mutant of S. macrospora FUS1. The latter carries no gfp gene. For interpretation of the fluorescence micrograph (below) the corresponding optical micrograph is reproduced above.

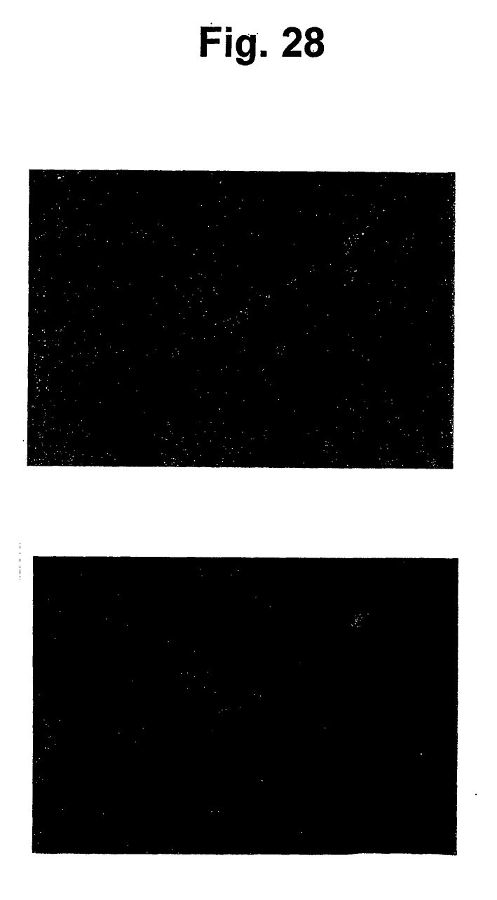

FIG. 28 shows micrographs of a transgenic S. macrospora strain which expresses the EGFP gene under the control of the ndk1 promoter. (a) Interference micrograph and (b) fluorescence micrograph.

### EXAMPLES

Material and Methods

The molecular biological and microbiological studies were carried out using standard methods according to the prior art (see, for example, Sambrook et al., 1989).

A. Cultivation of S. macrospora

1) Nutrient Media

BMM (fructification medium): 0.8% biomalt in corn meal extract, pH 6.5

BMM+NaAc (spore germination medium): BBB+0.5% NaAc, pH 6.0

CCM: 0.3% saccharose; 0.05% NaCl, 0.5% K₂HPO₄, 0.05% MgSO₄, 0.001% FeSO₄, 0.5% Tryptic Soy broth, 0.1% yeast extract, 0.1% meat extract, 1.5% dextrin pH 7.0

GM (minimal medium): 2% glucose, 0.7% Bacto Yeast Nitrogen Base, 0.4 μM biotin, 25 μl/L mineral concentrate (5% ascorbic acid, 5% ZnSO₄×7H₂O, 1% Fe(NH₄)₂(SO₄)₂×6H₂O, 0.25% CuSO₄×5H₂O, 0.05% MnSO₄×1H₂O, 0.05% H₃BO₄, 0.05% Na₂MoO₄, 1% chloroform, pH 6.0

GMU: GM with 10 mM uridine

GMS: GM with 10% saccharose

LB: 1% Bacto Trypton, 0.5% yeast extract, 0.5% NaCl, pH 7.2

CM: 0.15% KH₂PO₄, 0.05% KCl, 0.05% MgSO₄, 0.37% NH₄Cl, 1% glucose, 0.2% Trypton, 0.2% yeast extract, trace elements, pH 6.4-6.6

CMS: CM medium with 10.8% saccharose

MM (minimal medium): 55.5 mM glucose, 1.8 mM KH₂PO₄, 1.7 mM K₂HPO₄, 8.3 mM urea, 1 mM MgSO₄, 5 μM biotin, 0.1 ml/l mineral concentrate (see GM medium)

MMU: MM with 10 mM uridine

Solid media: 1.5% agar, GM Topagar 0.4% agar

2) Cultivation Conditions

Cultivation usually takes place at 27° C. For fructification S. macrospora is cultivated on solid media; fructification can already be observed after culture for 7 days. For nucleic acid preparations S. macrospora is cultivated on liquid media, usually in static cultures in Fernbach flasks.

B. Preparation of Sterile S. macrospora Strains

1) UV Mutagenesis

A protoplast suspension (1.5×10 protoplasts per ml protoplast buffer) of Sordaria macrospora wild type strain ATCC MYA-334 is prepared for the UV mutagenesis. The suspension is exposed to UV light (0.05 μW/cm²) whilst shaking gently. The times vary between 10 and 20 min. The protoplasts are then plated out on CMS solid medium (0.15% KH₂PO₄, 0.05% KCl, 0.05% MgSO₄, 0.37% NH₄Cl, 1% glucose, 0.2% Trypton, 0.2% yeast extract, trace elements, pH 6.4-6.6, 10.8% saccharose, 1.5% agar) and incubated for approx. 48 hours at 27° C. The regenerated protoplasts are then inoculated on MB solid medium (0.8% biomalt in corn meal extract, pH 6.5, 1.5% agar). After 1-4 weeks, phenotype characterization of the clones is carried out to identify sterile mutants. These are generally characterized by a modified fruiting body formation. In order to distinguish mutants from variants, the mitotic stability of the strains is tested by inoculation on MB medium. After a growth length of approx. 7 cm, the mycelium is retransferred to fresh nutrient medium. This process is repeated three times. After this test for mitotic stability, the sterile strains are tested genetically in crossover experiments in order to isolate ascospores. Homokaryotic strains are thereby produced.

2) EMS Mutagenesis

Ethyl methyl sulfonate is used as the mutagenic agent for the EMS mutagenesis. For the mutagenesis 5×10⁸ protoplasts of the wild type strain of Sordaria macrospora (see above) are treated with EMS in a total volume of 500 μl. The final concentration of EMS (GM-0880) is 34.1 mg/ml. The mutagenesis is carried out for 45 min at 27° C. The protoplasts are then plated out on CMS solid medium as under 1) and subjected to further treatment as described there.

The strains produced are characterized in the following by “inf” (infertile).

C. Transformation of Sterile S. macrospora Strains

The transformation of S. macrospora mutants is carried out exactly according to Walz and Kück (1995). Fungus mycelium of the strain to be transformed is cultivated as a static culture for 2 days at 27° C. in CM liquid medium (0.15% KH₂PO₄, 0.05% KCl, 0.05% MgSO₄, 0.37% NH₄Cl, 1% glucose, 0.2% Trypton, 0.2% yeast extract, trace elements, pH 6.4-6.6, 10.8% saccharose, 1.5% agar). After a mycelium washing step in protoplast buffer (13 mM Na₂HPO₄, 45 mM KH₂PO₄, 600 mM KCL, pH 6.0) and subsequent filtration, the mycelium is taken up into Novozym solution (10 mg Novozym 234 (Novo Nordisk)/ml protoplast buffer). After incubation for 45 min at 100 rpm and 27° C., the protoplasts are separated from the residual mycelium over a glass frit (pore size 1, Schott). After pelleting the protoplasts by centrifugation, the cells are washed twice in protoplast buffer and resedimented in each case. The protoplasts are then taken up into transformation buffer (1 M Sorbit, 80 mM CaCl₂, pH 7.5) so that the protoplast titer is 2×10⁸/ml. For the transformation, 2×10⁷/ml protoplasts are each mixed with 20 μg of plasmid DNA or 20 μl of Cosmid DNA and incubated for 10 min on ice. After adding 200 μl of 25% PEG (in transformation buffer), this is incubated for 20 min at room temperature. From the transformation formulation 100-200 μl in each case is plated out in different formulations directly onto the CMS medium. After incubation for a maximum of 12 hours at 27° C. the regenerating protoplasts are coated with hygromycin B-containing Topagar (0.8M NaCl, 0.8% agar). The hygromycin B concentration in the Topagar is selected so that a final concentration of 110 U/ml exists in the total medium. Transformands appear after 2 to 4 days and are inoculated on BMM medium (0.8% biomalt in corn meal extract, pH 6.5) containing 100 U/ml hygromycin B. The phenotype studies of the mutants are carried out on BMM medium without selection pressure.

D. Heterologous Gene Expression in Sordaria macrospora

For evidence of heterologous gene expression in Sordaria macrospora transformands of Sordaria macrospora together with the corresponding wild type control are transferred to CM medium and incubated at 27° C. Media or fungus mycelium are harvested by centrifuging or filtration.

### Example 1

### Comparison of the Transcriptional Expression of the ndk1, cpc2, pppg1 and acl1 Gene in Sordaria macrospora

In order to compare the transcriptional expression of the ndk1, cpc2, ppg1 and acl1 gene in Sordaria macrospora, the various transcription frequencies were determined for different strains in a Northern hybridization. For this purpose 5 μg of mRNA from the wild type strain or from the sterile mutant inf were applied and hybridized with the respective probes which were specific for the aforementioned genes. It can be seen from the autoradiogram shown in FIG. 1 that the ppg1 gene gives the weakest signals but the ppg1 gene is strongly transcribed compared with the so-called “house-keeping” genes (such as, for example, α or β Tubulin gene). The signal for the acl1 gene appears significantly stronger and the strongest signals were found for the cpc2 and the ndk1 gene. The autoradiogram clearly shows that the four genes are suitable for the construction of expression vectors because of their high transcriptional expression.

### Example 2

### Cloning of the Regulatory Sequences of the cpc2 and the ndk1 Gene of S. macrospora

For the cloning of the cpc2 and the ndk1 gene of S. macrospora a set of oligonucleotides was synthesized (see Table 2) which can be used for PCR amplifications. Using genomic DNA from S. macrospora the oligonucleotide primer 1095 and 1096 was used for the PCR amplifications of the cpc2 gene and the oligonucleotide primer 1265 and 1266 was used for the amplification of the ndk1 gene. The amplificates of 655 Bp (cpc2) or 594 Bp (ndk1) thereby produced were then used for the DNA sequencing. The DNA sequence of the ndk1 gene which also contains a 1.4 Kb promoter region before the putative ATG start codon is shown in FIG. 2. The ATG start codon is localized at the 1384-1386 position in this sequence. The nucleic acid sequence and the amino acid sequences of the cpc2 gene derived therefrom are shown in FIG. 3.

The gene fragments were then used for the so-called Northern hybridizations when comparative hybridizations with other genes of S. macrospora were carried out. It follows from the comparative hybridizations with 10 different S. macrospora gene probes that the cpc2 gene or the ndk1 gene of S. macrospora has a very high transcriptional level. This level should be classified as significantly higher compared with hybridization signals using other probes. In order to use this gene for the construction of expression vectors, the complete genomic copies of both genes were then isolated from an indexed genomic cosmid gene bank of S. macrospora (Pöggeler et al. 1997). The screening resulted in the isolation of the cosmid clones VIG10 (cpc2) and VIIG10 (ndk1). Subfragments of both cosmid clones were sequenced to uniquely identify the regulatory sequences.

### Example 3

### Subcloning of the cpc2 Promoter

For subcloning of the cpc2 promoter the corresponding cosmid clone was digested with EcoRV. A 3.0 kb fragment was identified which carries the cpc2 promoter. This fragment was inserted into the EcoRV linearized vector pBCKS+ (Stratagene, La Jolla, Calif.) in a shotgun experiment. The transformands obtained after transformation of E. coli with the recombinant vector were analyzed further by colony filter hybridization. Several positive clones could be identified among approx. 600 E. coli transformands. This finally resulted in isolation of a single clone which carries the recombinant plasmid pSE36-5. In the following control DNA sequencing it could be clearly proven that this recombinant plasmid carries parts of the cpc2 gene, and specifically the sequence from nucleotide position 1 to nucleotide position 2981 in FIG. 3.

The oligonucleotide pairs cpc9/cpc11 and cpc10/cpc12 were used for subamplification of parts of the cpc2 promoter from the plasmid pSE36-5. The oligonucleotide pair cpc9/cpc11 can be used for amplification of a 1359 bp amplicon (nucleotide positions 1250-2609 in FIG. 3) which as a result of the oligonucleotide sequence carries NcoI overhangs at both ends. The cpc10/cpc12 oligonucleotide pair could also be used to amplify a 1359 bp fragment and in this case EcoRV recognition sequences are generated at the ends of the fragment. The sequence of this fragment corresponds to the sequence from nucleotide position 1250 to nucleotide position 2609 in FIG. 3.

The amplificates described above were then subcloned into the vector pDrive (Qiagen, Hilden, GERMANY). After the corresponding ligation and transformation in E. coli, recombinant strains were identified by DNA hybridization. As a result, two recombinant plasmids having the following designation were obtained:

a) pSE38-16 contains an approx. 1.4 kb EcoRV fragment in the vector pDrive;

b) pSE39-14 contains an approx. 1.4 kb NcoI fragment in the vector pDrive.

### Example 4

### Construction of Expression Vectors with Regulatory Elements of the acl1 Gene of S. macrospora

In animals and fungi the ATP citrate lyase (ACL) is localized in cytosol whereas the homologous protein in plants is localized in chloroplasts. In all three organisms' systems ACL produces acetyl CoA which is primarily used for fatty acid and sterol biosynthesis. In fungi the enzyme consists of two subunits which are encoded by two separate genes (acl1, acl2) which are localized adjacently on the chromosomal DNA (Nowrousian et al. 2000). In contrast thereto, the continuous polypeptide in animals is encoded by one gene.

The promoter element of the acl1 gene of S. macrospora (Nowrousian et al. 1999) was used for the construction of the expression plasmid pMN110 (see FIG. 4). The oligonucleotides 1197 and 1199 (Table 2) together with the genomic DNA of S. macrospora were used as template DNA for amplification of the promoter sequence. The predicted 2.3 Kb fragment was cloned into the plasmid pMON 38201 (Borovkov and Rivkin, 1997). The resultant plasmid is designated pMN95. The terminator sequence of the acl1 gene was then amplified and cloned. In this case also, the genomic DNA ofS. macrospora was used as template DNA in order to generate a 0.6 Kb fragment by PCR with the oligonucleotides 1194 and 1200. The resultant plasmid is designated pMN102. The terminator sequence was then recloned from the plasmid pMN102 into the vector pKS+ (Stratagene, La Jolla, Calif.). For this purpose the plasmid pMN102 was hydrolyzed with the enzymes NotI and SacI. The resultant 0.6 Kb restriction fragment was ligated in the NotI and SacI restricted vector pKS+. The resultant plasmid is designated pMN109. This plasmid was then restricted with the enzymes HindIII and NotI and ligated with the 2.3 Kb fragment of the plasmid pMN95. The resultant plasmid is designated pMN110 and was used for further clonings. The cloning strategy is shown in FIG. 4 and the corresponding sequence of the insert DNA of the plasmid pMN110 is shown in FIG. 5.

The plasmid pMN112 was constructed in the next cloning step, this being suitable for the transformation of S. macrospora and for the transformation of E. coli (see FIG. 6). For this purpose the plasmid pBCHygro (Silar, 1995) was hydrolyzed with NotI and the corresponding restriction ends were filled in using Klenow polymerase. The resulting linear plasmid with filled-in NotI ends was restricted with the enzyme ClaI. As a result a linear vector molecule is formed which is terminated by a “blunt” end or by a ClaI cut. The vector molecule thus treated was used in a ligation in which a 2.9 Kb fragment from the plasmid pMN110 was used. This fragment was generated by linearizing the plasmid pMN110 with the enzyme HindIII. The overhanging restriction ends were then filled in with Klenow polymerase to generate “blunt” ends. The restriction fragment thus treated was then restricted with the enzyme ClaI and after gel electrophoresis, eluted for use for the ligation discussed above. The cloning strategy is shown in FIG. 6 and the resulting plasmid pMN112 is reproduced in FIG. 7. It has an overall size of 9.605 Kb.

The expression plasmid pMN112 can be used for the manufacture of heterologous proteins in S. macrospora. In this plasmid the acl1 promoter is linked to the acl1 terminator by an NotI restriction cut. This singular NotI restriction cut is suitable for the insertion of foreign DNA which should be expressed under the control of the acl1 promoter.

### Example 5

### Production of Bacterial β Galactosidase in Sordaria macrospora Under the Control of the acl1 Promoter

The plasmid pSMY1-1 was constructed in order to express the bacterial P galactosidase gene (lacZ) in S. macrospora. The plasmid pSMY1-1 was produced by inserting the lacZ gene into the singular NotI restriction site of the plasmid pMN112. The lacZ gene was generated from the plasmid pS18.8 (Menne et al., 1994) by PCR amplification. The oligonucleotides 1206 and 1215 (Table 2), which terminally have the recognition sequence for the NotI restriction enzyme, were used for this purpose. The amplificate has a size of 3.0 Kb and was inserted into the singular site of the plasmid PMON 38201 (Borovkov and Rivkin, 1997). The resulting plasmid has the designation pMN104 which was then hybridized with NotI. The resulting 3.0 Kb NotI fragment was inserted into the plasmid pMN112 linearized with NotI (see FIG. 7). The resulting plasmids pSMY1-1 (FIG. 8) and pSMY1-2 differ in respect of the orientation of the lacZ gene. In the pSMY1-1 plasmid the lacZ gene is under the control of the acl1 promoter. In pSMY1-2 there is an inverse arrangement of the lacZ gene with respect to the plasmid pSMY1-1 as a result of which no acl1 promoter controlled expression is possible. Thus, the plasmid pSMY1-2 can be used as a control in expression experiments. All the constructs formed were checked for their direction by control DNA sequencing. The sequence at the ATG start codon in the plasmid pSMY101 is reproduced in FIG. 9.

After transformation of Sordaria macrospora with the expression plasmids pSMY1-1 and pSMY1-2 the transformands selected on hygromycin were examined with regard to the formation of the heterologous gene product β galactosidase. For this purpose the fungus mycelium was mixed with glass beads and extraction buffer (2.5 mM tris-HCl (pH 8), 125 mM NaCl, 2 mM MgCl₂, 12 mM β mercaptoethanol (pH 7.5), 2 mM 4-methylumbelliferyl β-D-galactopyranoside, 10% (v/v) DMF) and macerated by intensive vortexing. The detritus was separated by centrifuging. Evidence of β galactosidase activity in the protein raw extract of the pSMY1-1 transformand is obtained by measuring the release of the fluorescing 4-methylumbelliferone from 4-methylumbelliferyl β-D-galactopyranoside. Whereas evidence of β galactosidase activity was obtained in the raw extract of the pSMY1-1 transformand, in the raw extract of the SMY1-2 transformand in which the expression cassette contains the lacZ gene in the inverse orientation, no β galactosidase activity could be detected.

### Example 7

### Production of the Pre-Protein of Human Serum Albumin Under the Control of the acl1 Promoter of Sordaria macrospora

In order to produce the pre-protein of human serum albumin (HSA) in Sordaria, the hsa gene (from the plasmid pPreHSA, Rhein Biotech GmbH, Düsseldorf, Germany) was cloned into the expression vector pMN112. For this purpose the gene for the pre-protein of human serum albumin (PreHsa) was obtained by amplification. Using the oligonucleotides hsa1 and hsa 2 the gene was amplified using the plasmid pPreHsa as template DNA. The 1.8 Kb amplificate has terminal NotI restriction sites. The PCR fragment was inserted into the Xcm1-restricted cloning vector PMON 38201 (Borovkov and Rivkin 1997) by ligation. The resulting plasmid is designated pMON-HSa. The insert of the plasmid pMON-HSA was checked by sequencing. This plasmid was then restricted with the enzyme NotI and the resulting 1.8 Kb fragment was inserted into the NotI-restricted vector pMN112. The plasmid thus obtained is designated pSMY4-1 (FIG. 10). The vector pSMY4-2 likewise formed by cloning contains the Pre-Hsa gene in inverse orientation and was used as a negative control for the expression experiments. All the constructs formed were checked for their accuracy by control DNA sequencing.

After transformation of Sordaria macrospora with the expression plasmids pSMY4-1 and pSMY4-2, the transformands selected on hygromycin were examined with respect to the formation of the heterologous gene product HSA. For this purpose the fungus mycelium was mixed with glass beads and extraction buffer (2.5 mM tris-HCl (pH 8), 125 mM NaCl, 2 mM MgCl₂, 12 mM β mercaptoethanol (pH 7.5)) and macerated by intensive vortexing. The detritus was separated by centrifuging. Evidence of HSA in the protein raw extract was obtained by means of an enzyme-linked immunosorbent assay (ELISA). The total protein extracts were pipetted into the cavities of a microtiter plate (MaxoSorp, Nunc) and incubated overnight at 4° C. After washing the plates three times with PBS buffer (137 mM NaCl, 2.7 mM KCl, 4.3 mM Na₂HPO₄, 14 mM KH₂PO₄, pH 7.4) containing 0.05% Tween® 20, the free binding sites were blocked for one hour using a 0.2% Tween® 20 solution in PBS buffer. After washing three times again, the HSA antibodies linked with peroxidase (BioTrend, Cologne, GERMANY; 1:1000 diluted in PBS buffer with 0.05% Tween® 20) were added and incubated for one hour at room temperature. The development of the ELISA was carried out by color reaction of the peroxidase substrate 3,3′,5,5′ tetramethyl benzidine (TMB) in the presence of hydrogen peroxide according to the manufacturer's data (Pierce, Helsingbirg). HSA could be detected in the raw extracts of the SMY4-1 transformands; in the expression controls using the starting strain and the SMY4-2 transformands the HSA proof was negative.

### Example 6

### Construction of Expression Vectors with Regulatory Elements of the ppg1 Gene of Sordaria macrospora

The ppg1 gene for the sexual pheromone of S. macrospora encodes a preproprotein of 277 amino acids. Included here is a leader peptide of 16 amino acids (Pöggeler, 2000) which can be used as a signal sequence for the protein secretion.

The promoter sequence, the leader-peptide coding sequence and the termination sequence of the ppg1 gene were used to construct the expression plasmid pSMY3 (see FIG. 11). The oligonucleotides ppg1-1 and ppg1-2 were used for the cloning of the promoter sequence together with the leader-peptide coding sequence. Both oligonucleotides contained sequences for restriction endonucleases (see Table 2). The recognition sequence for the enzyme SacI was used in the case of the oligonucleotide ppg1-1 and that for the enzyme NotI was used in the case of the oligonucleotide ppg1-2. The genomic DNA ofS. macrospora was used for the amplification with both these oligonucleotides. The amplification yielded a 1.8 Kb fragment.

The termination sequence of the ppg1 gene was also obtained by amplification of the corresponding sequence. The oligonucleotides ppg1-3 and ppg1-4 were used for this purpose. Both oligonucleotides also contained sequence extensions for the enzymes NotI (ppg1-3) or for the enzyme BamHI (ppg1-4). The amplification with both these oligonucleotides was again carried out using genomic DNA of S. macrospora and yielded an 880 Bp DNA fragment.

The two amplificates were inserted into the vector pMON38201 linearized with Xcm1 as described above. The plasmids resulting from the cloning were designated pPROM1 (contains the promoter region) or pTERM1 (contains the terminator sequence). The physical-genetic map of the plasmids pPROM1 and pTERM1 is shown in FIG. 12 and FIG. 13 respectively.

The promoter sequence was then recloned into the transformation vector pCB1004 (Carroll et al., 1994). For this purpose the plasmid pPROM1 was restricted with SacI and NotI and inserted into the SacI/NotI hydrolyzed vector pCB1004. The corresponding recombinant plasma is designated pSMY2 (FIG. 14). This plasmid was then hydrolyzed with the enzymes NotI and BamHI and the NotI and BamHI restriction fragment from the plasmid pTERM1 was inserted into the vector pSMY2 restricted with NotI and BamHI. The resulting plasmid is designated pSMY3 (FIG. 15) and contains both the promoter sequence including the leader peptide coding sequence and the terminator sequence of the ppg1 gene. The promoter sequence is linked to the terminator sequence by an NotI restriction cut. This restriction cut is singular in the plasmid pSMY3 and can thus be used for the insertion of heterologous DNA. The DNA sequence of the insert in the plasmid pSMY3 is shown in FIG. 16.

### Example 7

### Construction of Expression Vectors with Regulatory Elements of the cpc2 Gene of Sordaria macrospora

The construction of expression vectors with regulatory elements of the cpc2 gene is described in the following on the basis of the vector pSM2 already described. The vector pSM2 (see example 12, FIG. 26) contains the egfp gene which is fusioned with the TtrpC terminator of Aspergillus nidulans. Upstream of the egfp gene there is located a polylinker region which allows optimal cloning with shifted fragments.

In the first construct the EcoRV fragment described in Example 3 was ligated from the plasmid pSE38-16 into the vector pSM2/EcoRV. The resulting recombinant plasmid is designated pSE40-6 (FIG. 17). The correct orientation of the promoter fragment was checked by restriction analysis. In a second alternative vector the 1.4 Kb NcoI fragment from the plasmid pSE39-4 (see Example 3) was inserted into the vector pSM3 linearized with NcoI (see Example 12, FIG. 26). The resultant recombinant plasmid is designated pSE42-9. After restricting this plasmid with the enzyme SalI, the plasmid is linearized. This was followed by ligation with a 1.4 kb SalI fragment from the plasmid pCB1004. This SalI fragment carries the hygromycin B gene which can be used for selection in fungus transformands. The corresponding recombinant plasmid is designated pSE43-2 (FIG. 18).

In independent experiments the wild type strain of S. macrospora was transformed with the recombinant plasmids pSE40-6 and pSE43-2. After selection of the transformands on hygromycin B, approx. 20 transformands were isolated in each experiment. In the following analysis evidence was provided by fluorescence microscopic examinations that the heterologous egfp protein was produced in S. macrospora. In FIG. 19 the T1 p40-6- and T1 P43-2-transformands which carry the recombinant plasmid pSE40-6 or the recombinant plasmid pSE43-2 are shown as examples. The fluorescence is clearly and uniquely identifiable in the fluorescence microscope and is completely absent for the untransformed comparative strains (not shown). In the latter case no background fluorescence e.g., by phenolic substances, can be identified.

### Example 8

### Construction of the Base Plasmid pGC-MCS

In order to construct a vector which allows an exchange of promoter and terminator elements and selection markers and contains multicloning sequences (MCS), the starting vector pSMY5-1 (FIG. 20) was cut with BssHII and an approximately 4.6 Kb fragment was isolated by gel elution. The fragment was extended by a synthetic polylinker fragment and religated. The synthetic fragment was produced by hybridization of the following oligonucleotides. The oligonucleotide and the sequence of the restriction sites are given below. The BssHII sequence is underlined.

| | |
|---|---|
| Linker 1 | |
| 5′-TCGACGCGCGCCTCGAGAGGCCTACTAGTGAATTCAGATCTGGATCCGCGGCCGCA | (SEQ ID NO:24) |
| TCGATTCGCGAGGTACCGCGCGCA | |
| Linker 2 | |
| 5′-GCGCGCGGAGCTCTCCGGATGATCACTTAAGTCTAGACCTAGGCGCCGGCGTAGCT | (SEQ ID NO:25) |
| AAGCGCTCCATGGCGCGCGTTCGA | |

The sequence of the restriction sites in the synthetic cloning site was as follows: BssHII-AvaI-XhoI-StuI-SpeI-EcoRI-BglII-BamHI-SacII-NotI-SacII-NotI-ClaI-NruI-Acc65I-KpnI-BssHII. The construct obtained p-GV-MCS (FIG. 21) was checked by DNA sequencing.

### Example 9

### Construction of Expression Vectors with the ndk1 or cpc2 Promoter Based on the Base Plasmid pGV-MCS

In the following cloning steps various promoters and terminators were inserted into the vector pGV-MCS. Promoter elements were inserted at various sites of the MCS. For this purpose a 1378 bp fragment containing the ndk1 promoter (nucleotide positions 6-1383 in FIG. 2), a 1331 bp fragment containing the cpc2 promoter (nucleotide positions 1281-2611 in FIG. 3) and the terminator element of the acl1 gene (nucleotide positions 2338-2860 in FIG. 5) were either obtained from precursor plasmids with the aid of suitable restrictions or were amplified with terminal recognition sequences using PCR and cloned into the vector.

The oligonucleotides ndk5− (SpeI) and ndk− (EcoRI) (see Table 2) were used for PCR amplification of the ndk1 promoter with terminal SpeI and EcoRI sites. The oligonucleotides cpc2− (AvaI) and cpc2− (SpeI) (see Table 2) were used for PCR amplification of the cpc2 promoter with terminal SpeI and AvaI restriction sites.

In a next step the acl1 terminator was inserted into the vectors. The terminator element was obtained with terminal NotI/ClaI sites by PCR amplification from the plasmid pSMY1-2 (see example 5). The oligonucleotides acl1-(NotI) and acl1-(ClaI) (see Table 2) were used for amplification.

The vectors pGV-ndk1-MCS-acl1 (FIG. 22) and pGV-cpc2-MCS-acl1 thus obtained (FIG. 23) contain an MCS with the unique restriction sites EcoRI/BglII/BamHI/NotI to accommodate heterologous coding sequences.

### Example 10

### Production of Phytase in Sordaria macrospora Under the Control of the cpc2 or ndk1 Promoter

The phytase of Aspergillus fumigatus (Pasamontes et al., 1997) was selected as an example for the production of a secreted heterologous gene product. The coding region of the phytase gene as a 1403 Bp EcoRI fragment was cloned into the plasmid pGV-ndk1-MCS-acl1 (FIG. 22) downstream of the ndk1 promoter or into the plasmid pGV-cpc2-MCS-acl1 (FIG. 23) downstream of the cpc2 promoter. The resulting expression plasmids were verified with respect to their integrity and used for the transformation of Sordaria macrospora. Over a period of seven days the media supernatants were examined for secreting phytase activity. Corresponding aliquots were mixed with 25 μl of 5M NaAc and 50 μl of 4-nitrophenylphosphate. The formulation was incubated for 60 min at 37° C. The enzymatic conversion was stopped by adding 100 μl of 15% trichloroacetic acid. After adding 100 μl of 1M NaOH, positive culture supernatant samples appeared bright yellow. The yellow coloration was quantified by OD₄₀₅ measurement using a photometer.

The activity of the culture supernatants of the transformands with the respective phytase expression vector were determined in comparison to the wild type strain and a transformand with expression vector without phytase insert (mock transformand). Expression of the phytase gene in Sordaria macrospora and secretion of recombinant phytase in the culture supernatant could be proven both for the ndk1 and also for the cpc2 promoter.

For the expression of phytase controlled by the ndk1 promoter an OD₄₀₅ value of 0.4 was measured after 190 hours; the corresponding OD₄₀₅ values of the media supernatant of the Sordaria wild type or the mock transformand were 0.136 and 0.09 respectively. For the expression of phytase controlled by the cpc2 promoter an OD₄₀₅ value of 0.37 was measured after 190 hours; the corresponding OD₄₀₅ values of the media supernatant of the Sordaria wild type or the mock transformand were 0.049 and 0.05 respectively.

### Example 11

### Production of Human Lactoferrin in Sordaria macrospora Under the Control of the ndk1 or the cpc2 Promoter

A coding sequence for a fusion of human lactoferrin and the N-terminus of glucoamylase from Aspergillus awamori was cloned as a 3641 Bp EcoRI fragment into the vector pGV-ndk1-MCS-acl1 (FIG. 22). Restriction, fragment isolation and ligation took place under standard conditions. At the vector with the cpc2 promoter (pGV-cpc2-MCS-acl1, FIG. 23) which contains an additional EcoRI site in the promoter sequence, the EcoRI fragment was cloned into the BgIII/BamHI site. Blunt ends were produced at the fragment ends and the sites of the vector by Klenow treatment before the ligation. The treatment took place by standard methods.

Transformation with the generated vectors took place as described previously. Proof of positive lactoferrin-secreting transformands was provided by ELISA using the following protocol:

1. Coating a microtiter plate (Nunc Maxisorp) with 100 μl/sample well with anti-hlactoferrin in 0.1M NaCO₃ pH 9.6 (dilution 1:2400) (rabbit affinity-purified anti-hlactoferrin; ICN, Costa Mesa, Calif.; 2.4 mg/ml) and incubating overnight

2. Washing with 200 μl of PBS+0.05% Tween® 20 (3×)

3. Incubating with respectively 200 μl of PBS+0.05% Tween® 20+1% BSA for two hours at room temperature while shaking gently

4. Washing as in 2.

5. Application for 2 hours at room temperature of 100 μl/well of dilutions of an hlactoferrin standard(ICN). The dilutions are made in PBS (control) or in aliquots of culture supernatants of the various recombinant clones to be tested.

6. Washing as in 2.

7. Application of the second antibody for 1 hour at room temperature. Anti-hlactoferrin (peroxidase-conjugated) (rabbit affinity-purified; Jackson Immuno Research Laboratories, West Grove, Pa., USA) was used as the second antibody (1:5000 dilution of a 0.8 mg/ml solution in PBS+0.25% Tween® 20+0.25% BSA).

8. Washing as in 2.

9. Development of the TMB peroxidase substrate. TMB peroxidase substrate (Pierce, Helsingborg) and reaction solution (Pierce) were mixed in the ratio 1:1 and added to the sample wells in 100 μl aliquots. On reaching the desired color intensity, the reaction was stopped by adding respectively 100 μl of a 2M sulfuric acid solution.

10. The color intensity is measured at 450 nm in the ELISA reader. The concentrations are determined for the supernatant samples by comparison with the values in the standard series.

The ELISA measurements of the culture supernatants of the transformands with the respective lactoferrin expression vector were made in comparison to the wild type strain and a transformand with expression vector without lactoferrin insert (mock transformand). Expression of the lactoferrin gene in Sordaria macrospora and secretion of recombinant lactoferrin in the culture supernatant could be detected both for the ndk1 and for the cpc2 promoter. An OD₄₅₀ value of 1.375 was measured after seven days for the ndk1 promoter controlled expression of lactoferrin; the corresponding OD₄₅₀ values of the media supernatants of the Sordaria wild type or the mock transformand were 0.2 and 0.24, respectively. An OD₄₅₀ value of 1.95 was measured after seven days for the cpc2 promoter controlled expression of lactoferrin; the corresponding OD₄₅₀ values of the media supernatants of the Sordaria wild type or the mock transformand were 0.273 and 0.236, respectively.

### Example 12

### Construction of Expression Vectors and Production of “Green Fluorescent Protein” (GFP) in Sordaria macrospora Under the Control of the gpd Promoter of Aspergillus nidulans

In order to express the heterologous gfp gene (Clonetech, USA) in Sordaria macrospora, two expression plasmids were constructed for which the gfp gene was placed under the control of various promoters or is promoter-free. The plasmid pEGFP/gpd/tel was used as the starting plasmid (Inglis et al., 1999) (see FIG. 24).

In the pSM1 plasmid the gfp gene is controlled by the gpd promoter of Aspergillus nidulans and terminated by the trpc terminator sequence of Aspergillus nidulans. In addition, the plasmid contains the hygromycin B resistance gene for the selection of fungus transformands. The construction of the plasmid pSM1 is shown in FIG. 25. Unlike the plasmid pSM1, the plasmid pSM2 contains no gpd promoter. Before the gfp gene there is a multiple cloning site for a plurality of enzymes which are suitable for inserting heterologous or homologous promoter sequences and thus for controlling the gfp gene expression. The construction of the plasmid pSM3 is shown in FIG. 26.

A sterile Sordaria macrospora strain was transformed with the plasmids pEGFP/gpd/tel, pSM1 and pSM3 in three different experiments. The transformands obtained were then selected for hygromycin resistance as described and analyzed by fluorescence microscopy. The analysis was made using the Axiophot Zeiss microscope using exciting light at the 420 nm wavelength. GFP-producing clones were then used for a formal genetic analysis against the wild type strain or other tester strains. By crossing-over it can be checked to what extent the heterologous GFP protein is stably further inherited in the melose and in particular, how far the GFP expression is also retained stably in the descendants.

As can be seen from FIG. 27, the GFP gene expression can be identified both in the vegetative mycelium of the transformands and in the ascospores. The predicted 1:1 split is clearly identifiable in the eight-spore asci (4 spores show fluorescence, 4 spores show no fluorescence).

As a result of this GFP gene expression, it can also be made clear that after meiotic crossing-over the heterologous gene expression in S. macrospora is not destroyed by inactivation processes (e.g. RIP, MIP, Quelling).

### Example 13

### Production of GFP in Sordaria macrospora Under the Control of the cpc2 or ndk1 Promoter

The EGFP gene was amplified with the oligonucleotides EGFP5′ and EGFP3′ (see Table 2) by PCR from the vector pSM2. The PCR product obtained was cloned as a 726 Bp EcoRI fragment into the plasmid pGV-ndk1-MCS-acl1 (FIG. 22) downstream of the ndk1 promoter or into the plasmid pGV-cpc2-MCS-acl1 (FIG. 23) downstream of the cpc2 promoter. The resulting expression plasmids were verified with regard to their integrity and used for the transformation of Sordaria macrospora. The intracellular expression of the EGFP gene was proven as described in Example 12.

As an example, FIG. 28 shows a transformand which contains the EGFP gene under the control of the ndk1 promoter. The fluorescence attributable to EGFP was clearly identifiable in the fluorescence micrograph (below) and was completely absent (not shown) in the control (nontransformed strain).

TABLE 1
Comparison of the most commonly used amino acid codons in
E. coil, Saccharomyces cerevisiae (S.c.), Sordaria macrospora
(S.m.), Drosophila melanogaster (D.m.) and primates (Prim).
Those codons in the individual columns which are also used most
frequently with Sordaria macrospora are shown hatched.

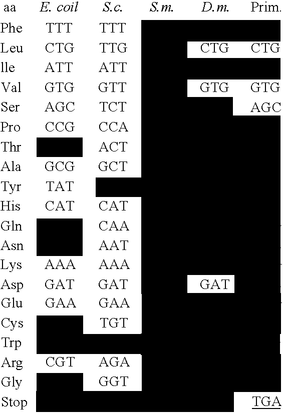

aa = amino acid

aa=amino acid

TABLE 2
Oligonucleotides used

| Oligo No. | SEQ ID NO: | Sequence 5′ -> 3′ | Characteristic |
|---|---|---|---|
| 1095 | 26 | CGCCGTTTCGTCGGCCACACC | cpc2 gene |
| 1096 | 27 | CGCAGAGCCAGTAGCGGTTGG | cpc2 gene |
| cpc9 | 28 | CCATGGTTGCAGTTCCTTTCT | cpc2 promoter |
| | | GGTTGATCA | NcoI overhang |
| cpc11 | 29 | CCATGGAGCATGATTGTTAAT | cpc2 promoter |
| | | GCGGAGAAG | NcoI overhang |
| cpc10 | 30 | GATATCTTGCAGTTCCTTTCTG | cpc2 promoter |
| | | GTTGATCA | EcoRV overhang |
| cpc12 | 31 | GATATCAGCATGATTGTTAAT | cpc2 promoter |
| | | GCGGAGAAG | EcoRV overhang |
| 1194 | 32 | GAGCTCATCGATCTCTTGTGCA | acl1 terminator |
| | | CCGTCAAAGTCCGG | ClaI/SacI |
| | | | overhang |
| 1197 | 33 | GCGGCCGCTAGTTGCGGAAGG | acl1 promoter |
| | | CATCTTC | NotI overhang |
| 1199 | 34 | AAGCTTCTAGAATGGCTTGGGC | acl1 promoter |
| | | TGCTTTGGCC | HindIII/Xbal |
| | | | overhang |
| 1200 | 35 | GCGGCCGCAAGGGAATTATATA | acl1 terminator |
| | | GGGATTAGGG | NotI overhang |
| 1206 | 36 | GCGGCCGCTTATTTTTGACACC | lacZ gene |
| | | AGACCAACTGG | NotI overhang |
| 1215 | 37 | GCGGCCGCGTCGTTTTACAACT | lacZ gene |
| | | GCTGTACTGGG | NotI overhang |
| 1265 | 38 | CCCCTCGGTTCTACCCGCC | ndk1 gene |
| 1266 | 39 | GAACCAGAGGGCAATCTCC | ndk1 gene |
| ppg1-1 | 40 | GAGCTCGCGAATCCTCCCGAAACC | ppg1 promoter |
| | | CA | SacI overhang |
| ppg1-2 | 41 | GCGGCCGCTAGCGATGGGCTGGGC | ppg1 promoter |
| | | AAC | NotI overhang |
| ppg1-3 | 42 | GCGGCCGCAAGCGCTATGCCTCCC | ppg1 terminator |
| | | CCG | NotI overhang |
| ppg1-4 | 43 | GGATCCTCTTGATCGCTATCCCCT | ppg1 terminator |
| | | TCT | BamHI overhang |
| hsa1 | 44 | GCGGCCGCAAGTGGGTAACCTTTA | hsa gene |
| | | TTTCCC | NotI overhang |
| hsa2 | 45 | GCGGCGCTTATAAGCCTAAGGCAG | hsa gene |
| | | CTTGA | NotI overhang |
| ndk5- | 46 | ATATTACTAGTGTGGTGGTGCACC | ndk1 promoter |
| (SpeI) | | TCG | SpeI overhang |
| ndk3- | 47 | ATATTGAATTCTTTGATTATGGGT | ndk1 promoter |
| (EcoRI) | | GGTTC | EcoRI overhang |
| cpc2- | 48 | GCTCGAGATCGGAAGGAGGTGGCG | cpc2 promoter |
| (AvaI) | | AG | AvaI overhang |
| cpc2- | 49 | CACTAGTCTTTGCAGTTCCTTTCT | cpc2 promoter |
| (SpeI) | | GGTTG | SpeI overhang |
| acl1- | 50 | GCGGCCGCAAGGGAATTAT | acl1 terminator |
| (NotI) | | | NotI overhang |
| acl1- | 51 | ATCGATCTCTTGTGCACCGT | acl1 terminator |
| (ClaI) | | | ClaI overhang |
| EGFP5′ | 52 | CGAATTCATGGTGAGCAAGGGCGA | EGFP gene |
| | | GG | EcoRI overhang |
| EGFP3′ | 53 | CGAATTCTTTACTTGTACAGCTCG | EGFP gene |
| | | TCCATG | EcoRI overhang |

TABLE 3
Recombinant plasmids

| Plasmid | |
|---|---|
| designation | Brief characterization |
| pSM1 | Bacterial base vector pBluescriptIIKS+ and the |
| | bacterial hygromycin B phosphotransferase gene |
| | (hph) under the control of the fungus promoter |
| | PtrpC of Aspergillus nidulans and in addition |
| | with the “enhanced green fluorescent protein |
| | gene” (EGFP) from Clonetech which is under the |
| | control of the promoter Pgpd of Aspergillus |
| | nidulans and the termination sequence TrpC of |
| | Aspergillus nidulans. |
| pSM2 | Like the vector pSM1 but without the Pgpd |
| | promoter before the EGFP gene. |
| pSM3 | Like the plasmid pSM2 but without the hph |
| | resistance gene |
| pMN110 | Bacterial base vector pMON38201 into which the |
| | acd1 promoter and the acl1 terminator were |
| | inserted. Promoter and terminator are |
| | fusioned by an NotI cut. |
| pMN112 | Vector pBCHygro + acl1 promoter and acl1 |
| | terminator as in the plasmid pMN110. |
| pPROM1 | Bacterial base vector pMON38201 + ppg1 |
| | promoter in SacI/NotI restriction sequence |
| pTERM1 | Bacterial base vector pMON38201 + ppg1 |
| | terminator sequence in NotI/BamHI restriction |
| | sequence |
| pSMY1-1 | As plasmid pMN112 + lacZ reporter gene under |
| | the control of the acl1 promoter |
| pSMY1-2 | As plasmid pSMY1-1, but lacZ with inverse |
| | orientation |
| pSMY2 | Shuttle vector pCB1004 + ppg1 promoter |
| | sequence inserted into the SacI/NotI |
| | restriction sequence |
| pSMY3 | As pSMY2 + ppg1 terminator sequence fusioned |
| | via NotI restriction sequence with the ppg1 |
| | promoter. |
| pSMY4-1 | As pMN112 + hsa gene under the control of the |
| | acl1 promoter |
| pSMY4-2 | As pSMY4-1, but inverse orientation of the hsa |
| | gene |
| pSMY5-1 | As pSMY3 + lacZ reporter gene under the |
| | control of the ppg1 promoter |
| pSMY5-2 | As pSMY5-1 but inverse orientation of the lacZ |
| | gene |

### References

Altschul S F et al. (1990) Basic local alignment search tool. J. Mol. Biol. 215: 403-410.

Borovkov A Y, Rivkin M I (1997) Xcm1-containing vector for direct cloning of PCR products. BioTechniques 22: 812-814

Carrol A M, Sweigard J A, Valent B (1994) Improved vectors for selecting resistance to hygromycin. Fungal Genet. Newsl. 41: 22

Cogoni C, Macino G (1999) Homology-dependent gene silencing in plants and fungi: a number of variations on the same theme. Curr. Opin. Microbiol. 2: 657-662

Devereux J et al. (1984) Nucleic Acids Research 12 (12): 387

Esser K, Straub J (1958) Genetic studies on Sordaria macrospora Auersw., Compensation and induction in cases of gene-induced development defects. Zeitschrift für Vererbungslehre 89: 729-746

Gordon C L, Archer D B, Jeenes D J, Doonan J H, Wells B, Tinci A Ü, Robson G D (2000) A glucoamylase: GFP gene fusion to study protein secretion by individual hyphae of Aspergillus niger. J. Microbiol. Methods 42(1): 39-48

Gouka R J, Punt P J, van den Hondel C A (1997) Glucoamylase gene fusions alleviate limitations for protein production in Aspergillus awamori at the transcriptional and (post) translational levels. Appl. Environ. Microbiol. 63(2): 488-597

Inglis P W, Queiroz P R, Valadares-Inglis M C (1999). Transformation with green fluorescent protein of Trichoderma Harzianum 1051, a strain with biocontrol activity against Crinipellis perniciosa, the agent of witches'-broom disease of cocoa. J. Gen. Appl. Microbiol. 45: 63-67

Kaster K R, Burgett S G, Roa R N and Ingolia T D (1983). Analysis of a bacterial hygromycin B resistance gene by transcriptional and translational fusions and by DNA sequencing. Nucleic Acids Res. 11(19): 6895-911

Le Chavanton L, Leblon G (1989). The ura5 gene of the ascomycete Sordaria macrospora: molecular cloning, characterization and expression in Escherichia coli. Gene 77: 39-49

Masloff S Pöggeler S, Kück U (1999). The pro1 gene from Sordaria macrospora encodes a C₆ zinc finger transcription factor required for fruiting body development. Genetics 152: 191-199

Menne S, Walz M, Kück U (1994). Expression studies with the bidirectional pcbAB-pcbC promoter region from Acremonium chrysogenum using reporter gene fusions. Appl. Microbiol. Biotechnol. 42: 57-66

Mullaney E J, Hamer J E, Roberti K A, Yalton M M, Timberlake W E (1985) Primary structure of the trpc gene from Aspergillus nidulans. Mol. Gen. Genet. 199: 37-45

Needleman and Wunsch (1970) J. Mol. Biol. 48: 443-453

Nowrousian M, Masioff S, Pöggeler S, Kück U (1999). Cell differentiation during sexual development of the fungus Sordaria macrospora requires ATP citrase lyase activity. Mol. Cell Biol. 19: 450-460

Nowrousian M, Kück U, Loser K. Weltring K M (2000). The fungal acl1 and acl2 genes encode two polypeptides with homology to the N- and C-terminal parts of the animal ATP citrate lyase polypeptide. Curr. Genet. 37: 189-193

Pasamontes L, Haiker M, Wyss M, Tessier M, van Loon A P G M (1997). Gene cloning, purification and characterization of a heat-stable phytase from the fungus Aspergillus fumigatus. Appl. Environ. Microbiol. 63: 1696-1700

Pöggeler S, Nowrousian M, Jacobsen S. Kück U (1997). An efficient procedure to isolate fungal genes from an indexed cosmid library. J. Microbiol. Meth. 29: 49-61

Pöggeler S (2000). Two pheromone precursor genes are transcriptionally expressed in the homothallic ascomycete Sordaria macrospora. Curr. Genet. 37: 403-411

Sambrook J, Fritsch E F, Maniatis T (1989). Molecular cloning: a laboratory manual. 2nd edition, Cold Spring Harbor Laboratory, Cold Spring Harbor, N.Y.

Selker E U (1997). Epigenetic phenomena in filamentous fungi: Useful paradigms or repeat-induced confusion? Trends Genet. 13(8): 296-301

Silar P C (1995). Two easy to use vectors for transformations. Fungal Genet. Newsl. 42: 73

Singer M J, Marcotte B A, Selker E U (1995) DNA methylation associated with repeat-induced point mutation in Neurospora crassa. Mol. Cell Biol. 15(10): 5586-5597

Turcq B and Begueret J (1987). The ura5 gene of the filamentous fungus Podospora anserina: nucleotide sequence and expression in transformed strains. Gene 53: 201-9

Walz M, Kück U (1995). Transformation of Sordaria macrospora to hygromycin B resistance: characterization of transgenic strains by electrophoretic karyotyping and tetrad analysis. Curr. Genet, 29: 88-95

1. A method for the production of heterologous protein in a filamentous fungus, comprising
the cultivation of a sterile mutant of a homothallic fungus of the family Sordariaceae, which contains an expression cassette which contains the following elements in functional combination:
a promoter active in the fungus of the family Sordariaceae,
a heterologous gene and
a terminator active in the fungus of the family Sordariaceae,
and the harvesting of the protein produced in an inherently known fashion.

2. The method according to claim 1, characterized in that the homothallic fungus belongs to the genus Sordaria.

3. The method according to claim 2, characterized in that the homothallic fungus is Sordaria macrospora or Sordaria fimicola.

4. The method according to any one of claims 1-3, characterized in that the cultivation of the homothallic fungus takes place at 27±2° C.

5. The method according to any one of claims 1 to 4, characterized in that the promoter active in the homothallic fungus of the family Sordariaceae originates from a filamentous fungus.

6. The method according to claim 5, characterized in that the promoter is a promoter from Sordaria macrospora.

7. The method according to claim 6, characterized in that the promoter is the acl1 promoter, the ppg1 promoter, the cpc2 promoter or the ndk1 promoter from Sordaria macrospora.

8. The method according to any one of claims 1 to 7, characterized in that the terminator active in the fungus of the family Sordariaceae originates from a filamentous fungus.

9. The method according to claim 8, characterized in that the terminator is a terminator from Sordaria macrospora.

10. The method according to claim 9, characterized in that the terminator is the acl1 terminator, the ppg1 terminator, the cpc2 terminator or the ndk1 terminator from Sordaria macrospora.

11. The method according to any one of claims 1 to 10, characterized in that the heterologous gene encodes a protein glycosylated after expression in eukaryonts.

12. The method according to any one of claims 1 to 11, characterized in that the heterologous gene is a growth factor, a cytokine, a clotting factor, an industrial protein or a technical enzyme.

13. The method according to claim 12, characterized in that the heterologous gene encodes one of the following proteins: G-CSF, GM-CSF, IL-1, IL-2, IL-4, IL-6, IL1ra, IFN-alpha, IFN-beta, IFN-gamma, erythropoietin, glucoamylase, clotting factor VIII, clotting factor XII, clotting factor XIII, human serum albumin.

14. The method according to any one of claims 1 to 13, characterized in that between the promoter and the heterologous gene in the reading frame with the heterologous gene there is arranged a sequence which encodes a signal sequence which functions in the fungus of the family Sordariaceae.

15. The method according to claim 14, characterized in that the signal sequence is a signal sequence from a filamentous fungus.

16. The method according to claim 15, characterized in that the signal sequence is a signal sequence from Sordaria macrospora.

17. A nucleic acid molecule, comprising:
(1) a promoter active in a homothallic fungus of the family Sordariaceae, which is selected from the following nucleic acids:
(a) a nucleic acid having the sequence specified in SEQ ID NO:1;
(b) a nucleic acid having the sequence specified in SEQ ID NO:2;
(c) a nucleic acid having a sequence which exhibits at least 90% identity with one of the sequences specified in (a) or (b);
(d) a fragment of one of the nucleic acids specified in (a) to (c) which retains the function of the promoter active in the fungus of the family Sordariaceae;
(e) a combination of a plurality of nucleic acids specified in (a) to (d) wherein the sequences of the nucleic acids can be the same or different; or
(2) a nucleic acid having a sequence which is complementary to the sequence of one of the nucleic acids specified in (a) to (e).

18. The nucleic acid molecule according to claim 17, characterized in that the nucleic acid specified under (c) exhibits at least 95% identity with one of the sequences specified in (a) or (b).

19. A vector for the transformation of a sterile mutant of a homothallic fungus of the family Sordariaceae, characterized in that the vector contains the following elements in functional combination one with the other:
a promoter active in the fungus of the family Sordariaceae,
a heterologous gene,
a terminator active in the fungus of the family Sordariaceae as well as
a selection marker.
an active promoter which comprises a nucleic acid according to any one of claims 17 to 18, alternative (1) or which is the acl1 promoter from Sordaria macrospora or the ppg1 promoter from Sordaria macrospora.

20. The vector according to claim 19, characterized in that the terminator active in the family Sordariaceae is the acl1 terminator, the ppg1 terminator, the cpc2 terminator or the ndk1 terminator from Sordaria macrospora.

21. The vector according to any one of claims 19 or 20, characterized in that the selection marker is a hygromycin B-resistance gene.

22. A host organism characterized in that it is a sterile mutant of a homothallic fungus of the family Sordariaceae which contains a vector according to any one of claims 19 to 21.

23. The host organism according to claim 22, characterized in that it belongs to the genus Sordaria.

24. The host organism according to claim 23, characterized in that it is Sordaria macrospora or Sordaria fimicola.

25. A kit comprising:
(a) a vector according to any one of claims 19 to 21 and
(b) a sterile mutant of a homothallic fungus of the family Sordariaceae suitable for the production of heterologous protein.

26. The use of a nucleic acid molecule according to any one of claims 17 to 18 or an expression vector according to any one of claims 19 to 21 or a kit according to claim 25 for the expression of a heterologous gene under the control of the promoter.

27. The use of a nucleic acid molecule according to any one of claims 17 to 18 or an expression vector according to any one of claims 19 to 21 or a kit according to claim 25 for the production of one or a plurality of proteins.


SEQUENCE LISTING (53 sequences)

SEQ ID NO: 1 - DNA, 1377 bp, Sordaria macrospora
  ndk1 promoter region
tggtggtgcacctcgctgaggttagaactcgctaagaaagtcaaaagaaacaccaagcaagaacagacaagcaacaacaaaaggggactggttagaagctcgtctgggtcatcaagaacaacactcccgggtttgctaaggtatgtcgcagagtcccttgaagctgtcgtcgactgcagtatactccgcagctgtgtaaggtaaggtaggtagaggtaatgtaagcggaagtcacgagttgggttgacctgttgtcgtcaatggcaatcaatgcctgccgtgttggcttcaagatggcagttgttagctggcagcatcgcaacaagtccacatctcggtggccgatggcgtcgagtggccccaattcgccccgtggaagccaagcaggccaagctgtcccctgccagctagcgcaccgcttggcatcgcgggattcgttagatgcatagtggctctagatgataaaccgcggggtaagttttgatatctgcatgacgacctcagcccaccgtctcagttctcagggcctgtagtgtacaactgacttgggcgaacatatgttcctctaatcagttgacctggttgttcgtgattaatatggcattcgaggggaatgacttcctttaaatattgaagagcattgggggttatctcggaaagaaaacctcagctgcacaggccgtacagattcaatgggggggagctctgcgagctttgctggcacccatcgtggcttctcgtctttccggcctcgtttccgagacgttgcgcaaccacttaaacagagacccataagaccaattgacggacggctgcctggtcgcttgctgttctctgctccatcaaaacattgccctaatttcttcaaacccgtcgcgtagagttgtcagctgtttgctgtcctgctgctgagtctggaccaacattcttctaaaaatcagagctttgagctaagctttcatcacctcaccaactgaaatagctgcatccaataagaccatgtcgaacactgacagctcggaacatctgcgacgagaatcagacggacaacgcagaccgctgccgcaaactgaagtgcaacagaagcagcccgaatcgggaggcgcaatggctgcgggtgctgattctagtggtgcccaatctcgccaatcgtcccaatcgaggggaagggggagctccagaaggagacgagccaggtcacccaggggaacgggcccaccaattttttttccggccgcgtgtggactcctcctagcagtcctccttttctatctcggccgcctctcctcgtcttcctgctcgacttctccttctccatcatcaatcatcatcccatcacaaatctatactccctcacaggaaccacccataatcaaa

SEQ ID NO: 2 - DNA, 2611 bp, Sordaria macrospora
  cpc2 promoter region
gatatcccatacggccgctgaacgaacctttgtcagtacggattagcgaccgatataaataccatcgaccaaacgtgttctgcctggctttgtccccagctctcgcgaacaacatcccagcagcccagccatggcgaccgacacgcgcaagagactggcggacgcagagggagcgcaaccgtcaccagccaaacgaccaaaaaccaacacatcatcacgaactattctcgcagtcgaagccatcggaaagtcaccaaatgcatcgacaacacccacacccgacgaactggctaagaatggactgaggcggacgattgcgttggcgctggaaacggtcggcttcgacggagccacgcctgaagcattagagagcttcaccctaatgacggagacgtgtacgccgcctgttgtgccgaggctgccccgcgtcaccttggcttcatgctaacctcacctttgtgggcagacctccattcattaaccgaagaagtcaagatctttgccaacgccgcgcgccgctcctacccgatactcaatgacttcgacaacacactgaagcgcttcaacctcacaccaacagccctctattatcacaggaaacccccggtaccgaagaagaagcgacagccagtgtgggaagatctcccctcggcgaagccatcccaccgacctccccgtcctcgctcccgaactcgacggcgccgcagacaaggcagcgaagaactacatcccgagctccttcccctccttccccagcgtgcacacctacaaatacacgcccgaaagcgtcgaggccgccacggccgtagacgacagcagcatcataaccgacacacaagccacaaccgccacgcagacccaaacgatgatcgcagaatcccagaccgaagcagcagtagcagcgttagcatcccagcaacaacaatcacaactccccaccaagatcccccaacgaccactggcgcccgacgagatcccccgcggcgatcctaagaagatccgcgaagccgccgccaaggaagccaaggccggcgagcaagctctacgtcgcctcatgcgcgccagcaagattgccaaacagaaggacgtgtcggccacggcgcagcgggcgccggccaaacgcgagcggtacgcgctctgggaagcggccatgcgcgagctggtcgaggacgatagcaaggccaagggtagagaggtgacggctgtggctatgcacagtcagcagggccgggtcgagattgcggatcacagcatgattgttaatgcggagaaggggtactatcggaaggaggtggcgaggccgggggtaatgaagttggcgggggagttggcggggaagtagacattggcgggagggtacgagggagatggtacgagggcggtaatgatgaaggtggctaccagggaagaggtcatgttgaggaattatctctttcggtggctgaaggatgacgagttcaccatgtggaaatggtaaagatcaaaaatggctatgatcatggccgtggtcttgcgaggaaattgcacatggaggctggagagattggcatacgaagagaggagtgtggtgtcagccttgaaagatggcaccgagctgtcattgcttggttcaaaaggcatgaggagttagggaaggcgtatcattgatagcattagcatttgtcgtctttctcaagtttacaagatacccatatgaaacatacaaatagacgagcagtggtctatatagcggatgttgcttggtttcggtgttatgcaatgacttggtacattttgggtatcgtccttaaggggttcacattaaggagaaaacacgggactgaatttccaagaagctacagagggagcaaagagtctatggacgtccatgcgtgtgttgaagtcaatattgattattatgaacgactgagatgcgttttgcaaacagagctcttcaataactcgaatctgccagtaaatgtcctagtaaaagggcaatttgaagtcttgtcagttcgagtttaataccacctattggcaccaattgcataaacaaaccaaacagaactgtcatgtgaaggtctgcatacttttgtgcacaggtacctactgtgtatgagtgaagtcatcgtcgaaccttggacgaatctgtttgtcatcgccgaggaagtgaagctccaaagtacacaactaaagctgccagcttttgccgggccggcaacggcacagtgcacttggagtgacgacactgcagttccgggcaggcctggccctccaatttggtgaagtgtggcagacagtgtgcaatttgcgaccagccgtttgaggccacaggaggaaagcgccaaggcggcaacagcccaaagagccccaccacgcaccacgcacccagcgaccgccgccgcagtgcctggctgtggcagctgtggcaggcgggctgcccgcgtcggccgaccctattttccgccgcacaaaaatttccggaattcctacctggtcccgacgccccagtctccgcattgtgccttttgacgattttctgacttcccgcgcagagaggcaccgtgaaaaagctcaccaccccatttcccccattgatcctcccctgatcaaccagaaaggaactgcaaag

SEQ ID NO: 3 - DNA, 1853 bp, Artificial sequence
  Description of the artificial sequence fragment of the ndk1 gene with a XhoI restriction site at the 5′ terminus
ctcgagtggtggtgcacctcgctgaggttagaactcgctaagaaagtcaaaagaaacaccaagcaagaacagacaagcaacaacaaaaggggactggttagaagctcgtctgggtcatcaagaacaacactcccgggtttgctaaggtatgtcgcagagtcccttgaagctgtcgtcgactgcagtatactccgcagctgtgtaaggtaaggtaggtagaggtaatgtaagcggaagtcacgagttgggttgacctgttgtcgtcaatggcaatcaatgcctgccgtgttggcttcaagatggcagttgttagctggcagcatcgcaacaagtccacatctcggtggccgatggcgtcgagtggccccaattcgccccgtggaagccaagcaggccaagctgtcccctgccagctagcgcaccgcttggcatcgcgggattcgttagatgcatagtggctctagatgataaaccgcggggtaagttttgatatctgcatgacgacctcagcccaccgtctcagttctcagggcctgtagtgtacaactgacttgggcgaacatatgttcctctaatcagttgacctggttgttcgtgattaatatggcattcgaggggaatgacttcctttaaatattgaagagcattgggggttatctcggaaagaaaacctcagctgcacaggccgtacagattcaatgggggggagctctgcgagctttgctggcacccatcgtggcttctcgtctttccggcctcgtttccgagacgttgcgcaaccacttaaacagagacccataagaccaattgacggacggctgcctggtcgcttgctgttctctgctccatcaaaacattgccctaatttcttcaaacccgtcgcgtagagttgtcagctgtttgctgtcctgctgctgagtctggaccaacattcttctaaaaatcagagctttgagctaagctttcatcacctcaccaactgaaatagctgcatccaataagaccatgtcgaacactgacagctcggaacatctgcgacgagaatcagacggacaacgcagaccgctgccgcaaactgaagtgcaacagaagcagcccgaatcgggaggcgcaatggctgcgggtgctgattctagtggtgcccaatctcgccaatcgtcccaatcgaggggaagggggagctccagaaggagacgagccaggtcacccaggggaacgggcccaccaattttttttccggccgcgtgtggactcctcctagcagtcctccttttctatctcggccgcctctcctcgtcttcctgctcgacttctccttctccatcatcaatcatcatcccatcacaaatctatactccctcacaggaaccacccataatcaaaatgtccaaccaggagcagacgtaagttattttttactacaacacgggaattcaaacccctcggttgtaccccgccatctttattgccgtctgttacgctcgcgcccatcatcaacagaagcgccagcaacaccatcacccagcactactcacaatgcaacagagtcgattgactaacttgcgcctctaacagcttcattgccgtcaagcccgatggcgtccagcgtggcctcgttggcaacatcgtctctcgcttcgagaaccgcggcttcaagctcgttgccatgaagctcaccagccccggccaggcccacctcgagaagcactacgaggacctcaacaccaagcccttcttcgctggcctcatcaagtacatgaactccggccccatctgcgccatggtctgggagggcaaggacgccgtcaagaccggccgcaccatcctcggtgccaccaaccccctcgcctccg

SEQ ID NO: 4 - DNA, 4540 bp, Sordaria macrospora
  cdc2 gene
gatatcccatacggccgctgaacgaacctttgtcagtacggattagcgaccgatataaataccatcgaccaaacgtgttctgcctggctttgtccccagctctcgcgaacaacatcccagcagcccagccatggcgaccgacacgcgcaagagactggcggacgcagagggagcgcaaccgtcaccagccaaacgaccaaaaaccaacacatcatcacgaactattctcgcagtcgaagccatcggaaagtcaccaaatgcatcgacaacacccacacccgacgaactggctaagaatggactgaggcggacgattgcgttggcgctggaaacggtcggcttcgacggagccacgcctgaagcattagagagcttcaccctaatgacggagacgtgtacgccgcctgttgtgccgaggctgccccgcgtcaccttggcttcatgctaacctcacctttgtgggcagacctccattcattaaccgaagaagtcaagatctttgccaacgccgcgcgccgctcctacccgatactcaatgacttcgacaacacactgaagcgcttcaacctcacaccaacagccctctattatcacaggaaacccccggtaccgaagaagaagcgacagccagtgtgggaagatctcccctcggcgaagccatcccaccgacctccccgtcctcgctcccgaactcgacggcgccgcagacaaggcagcgaagaactacatcccgagctccttcccctccttccccagcgtgcacacctacaaatacacgcccgaaagcgtcgaggccgccacggccgtagacgacagcagcatcataaccgacacacaagccacaaccgccacgcagacccaaacgatgatcgcagaatcccagaccgaagcagcagtagcagcgttagcatcccagcaacaacaatcacaactccccaccaagatcccccaacgaccactggcgcccgacgagatcccccgcggcgatcctaagaagatccgcgaagccgccgccaaggaagccaaggccggcgagcaagctctacgtcgcctcatgcgcgccagcaagattgccaaacagaaggacgtgtcggccacggcgcagcgggcgccggccaaacgcgagcggtacgcgctctgggaagcggccatgcgcgagctggtcgaggacgatagcaaggccaagggtagagaggtgacggctgtggctatgcacagtcagcagggccgggtcgagattgcggatcacagcatgattgttaatgcggagaaggggtactatcggaaggaggtggcgaggccgggggtaatgaagttggcgggggagttggcggggaagtagacattggcgggagggtacgagggagatggtacgagggcggtaatgatgaaggtggctaccagggaagaggtcatgttgaggaattatctctttcggtggctgaaggatgacgagttcaccatgtggaaatggtaaagatcaaaaatggctatgatcatggccgtggtcttgcgaggaaattgcacatggaggctggagagattggcatacgaagagaggagtgtggtgtcagccttgaaagatggcaccgagctgtcattgcttggttcaaaaggcatgaggagttagggaaggcgtatcattgatagcattagcatttgtcgtctttctcaagtttacaagatacccatatgaaacatacaaatagacgagcagtggtctatatagcggatgttgcttggtttcggtgttatgcaatgacttggtacattttgggtatcgtccttaaggggttcacattaaggagaaaacacgggactgaatttccaagaagctacagagggagcaaagagtctatggacgtccatgcgtgtgttgaagtcaatattgattattatgaacgactgagatgcgttttgcaaacagagctcttcaataactcgaatctgccagtaaatgtcctagtaaaagggcaatttgaagtcttgtcagttcgagtttaataccacctattggcaccaattgcataaacaaaccaaacagaactgtcatgtgaaggtctgcatacttttgtgcacaggtacctactgtgtatgagtgaagtcatcgtcgaaccttggacgaatctgtttgtcatcgccgaggaagtgaagctccaaagtacacaactaaagctgccagcttttgccgggccggcaacggcacagtgcacttggagtgacgacactgcagttccgggcaggcctggccctccaatttggtgaagtgtggcagacagtgtgcaatttgcgaccagccgtttgaggccacaggaggaaagcgccaaggcggcaacagcccaaagagccccaccacgcaccacgcacccagcgaccgccgccgcagtgcctggctgtggcagctgtggcaggcgggctgcccgcgtcggccgaccctattttccgccgcacaaaaatttccggaattcctacctggtcccgacgccccagtctccgcattgtgccttttgacgattttctgacttcccgcgcagagaggcaccgtgaaaaagctcaccaccccatttcccccattgatcctcccctgatcaaccagaaaggaactgcaaagatggctgagcaactcatcctcaagggcaccctggagggccacgtaagtttttacaatttcttgcgaatttccctccccccgactcgctgacgttgctgtttgctgcttgctgctgttgctgccatgttgtcgatgttgcgctggtgggtggacttgacaattgaggagggagcactgcaatgggcgcaattgaaaagagggtattactgcgacgacggcgacaacgacaacttgggcaggcgacgacaatggcgacatctacatggcagcaggaaaagcagacagcaacaaggcgattcgtgaaccaccacacattcgagatatgtggtgggaaaagtataaatgaattatacagaaaactgaccatgatatcttctacagaatggctgggtcaccagcttggccacctctttggagaagtacgaaatcaccgaatactcgcgcgaaagatcatgaacttacaaatgctctcgcaattagccccaacatgctcctttctggtagcagagacaagtccctcatcatctggaacctcacccgcgatgagacctcgtacggctaccccaagcgccgtctccacggccactctcacatcgtctccgactgtgtacgttaattctcgatggacacgaattgggcggaggatttgttgatatctgggaacatatcgggggcagtttgactgattggcgcggcacaggtgatctcttccgacggtgcctacgccctctctgcctcgtgggacaagaccctccgtctctgggagctttccaccggcaccaccacccgccgtttcgtcggccacaccaacgacgttctctccgtcagcttctccgccgacaaccgtcagatcgtctccggttcccgcgatcgctccatcaagctctggaacaccctcggtgactgcaagttcaccatcaccgagaagggccacaccgagtgggtttcttgcgtccgcttcagccccaacccccagaaccccgtcattgtctcctccggctgggacaagctcgtcaaggtatgttgtaatctatcacactccccaaatccgacaccatgatgtttgcatatttttttcaaaaaatagtatttgctacttcagagccctcgggccatgagaccaacttcacacgcactgattttctcggaaatcataactccctgttattcatcacacatggaatttccgtggatcttgcgactaaccaagtcacaaacaggtttgggagctctcgtcctgcaagctccagactgaccacatcggtcacaccggctacatcaacgccgtcaccatctcccccgatggctccctctgcgcctccggtggcaaggacggtaccaccatgctctgggacctcaacgagagcaagcacctctactctctcaacgccaacgacgagatccacgccctcgtcttctcccccaaccgctactggctctgcgctgccacctccagcagcatcatcatcttcgatctcgagaagaagagcaaggtcgacgagctcaagcccgagttccagaacatcggcaagaagtcccgcgagcccgagtgcgtctccctcgcctggtccgccgatggccagaccctcttcgccggttacaccgacaacatcatccgtgcctggggtgtcatgtcccgcgcttaagcgtttgagacgtagtcgtcggagccggaactggttggctaggggacgttgtggacgggaggcttcatgtgcggcggcataacgtgttggcagttagtccgtgaaggacgacggacgcctcgcttgcgtgtgtcgtgctcaggctgcatcagggcaatttcgttcagggattcttcttccatggtaaaaggcgtatctcggaaactgctttgtgtatcacgtctttgctttctttcattttaaaacataccaaaatgaaaggacaactgggagctatgggaaa

SEQ ID NO: 5 - protein, 14 aa, Sordaria macrospora
MAEQLILKGTLEGH

SEQ ID NO: 6 - protein, 13 aa, Sordaria macrospora
NGWVTSLATSLEN

SEQ ID NO: 7 - protein, 42 aa, Sordaria macrospora
PNMLLSGSRDKSLIIWNLTRDETSYGYPKRRLHGHSHIVSDC

SEQ ID NO: 8 - protein, 106 aa, Sordaria macrospora
VISSDGAYALSASWDKTLRLWELSTGTTTRRFVGHTNDVLSVSFSADNRQIVSGSRDRSIKLWNTLGDCKFTITEKGHTEWVSCVRFSPNPQNPVIVSSGWDKLVK

SEQ ID NO: 9 - protein, 141 aa, Sordaria macrospora
VWELSSCKLQTDHIGHTGYINAVTISPDGSLCASGGKDGTTMLWDLNESKHLYSLNANDEIHALVFSPNRYWLCAATSSSIIIFDLEKKSKVDELKPEFQNIGKKSREPECVSLAWSADGQTLFAGYTDNIIRAWGVMSRA

SEQ ID NO: 10 - DNA, 48 bp, Artificial sequence
  Description of the artificial sequence transition of the acl1 promoter/acl1 terminator of the plasmid pMN110
gaagatgccttccgcaactagcggccgcaagggaattatatagggatt

SEQ ID NO: 11 - DNA, 48 bp, Artificial sequence
  Description of the artificial sequence transition of the acl1 promoter/acl1 terminator of the plasmid pMN110; complementary strand
aatccctatataatacccttgcggccgctagttgcggaaggcatcttc

SEQ ID NO: 12 - protein, 12 aa, Artificial sequence
  Description of the artificial sequence amino acid sequence encoded by the transition of the acl1 promoter/acl1 terminator of the plasmid pMN110;
MPSATSGRKGII

SEQ ID NO: 13 - DNA, 2866 bp, Artificial sequence
  Description of the artificial sequence nucleotide sequence (promoter and terminator) of the plasmid pMN110
aagcttctagaatggcttgggctgctttggccgcttgttctgtggcattacaacagatgcccgatgttttactgtttagtgttttcttttacccccaaacccgagtgggctttccatgatattcgtttacctattcctcttccaaaaaaagaccccaagtggcccagaaacccactggttgagtcacggtggatgctgtcgacgatccagtgatccaagcgcaagctggaaccccaaagacgggggcggcgtggcgctcgtcaattacgatgtggaatggccgtgacggacaggacagtctcatttccaatcacttattcttgtccctccgcgcctcttgggcgggcccgtagttggggatcgtccggtgccggctcccacttacacgcttgtctgtgggttgactccagtttcccctcaagcccctctcgtgactgtttggtccaagtctaaaagccttgtatgatggagacagactggagtacgggacaagccacgggattccgtagtggagagacaatcgacctggaaaagaaagcccgttgtcgatgttgttctcttctccaagccggaaagcccctttttttgtttcgctgttgttgtcttcagttcatgtttctttgtggttggctcccccacattgaggtggcccaatcggtctccaagtggcacgcacatcttaccccgatgtttaatggcaatggctgcaacctccctccctgccaccaacgccttcaggcacggacaaactctgattattgatcatgactatattcgggtcctgttagtcctcttgacccccggcctcgtcgtgcacatcgaggccagtctgcccctattctgctttccgtttcgagaagcacaaggtgttctttacagatcaattactttaggcgattgtctaaggtaatggcatgcacttgccaagcgatgccaagggccacgaaaggaccgcagcttcatacctttgtcgacgccgagtggaactagaaaccccccgaggacccgcgggcgccgccttccacaggggtagaggtaggggtaatggccgctttccaggggaccaccagatagggcaagtctcgagtggacccccttcggtcacatcaagctgtgtggatgacaccgttgcccggcacccagagctctgatgtttggagtgaagatctcttgcttgatatctccaaaagctatgatgacagttcattgcatgataaggttggatgacagctagttgctacacccaagacctctagtcggttgctgcttaggggcaggctgccatttacactcttattataaacatttcctatgtcatgttgacttcctggaggatgggaaggattactcttttgcttatggaggggtcatcatggtgcacccaaagaataaacatccggactgtctccagctgtgtctgtggtcgggtagccatgtcggagacggcgagcattccaatgccaacacgcaatggcaatgaacatggaccacggttgccacggcccatctgcgcctatcaggatcggatgaggtgttgcatgcaagttacccttggcggtaccggtacttaggatcaccaagctgcgggtggggcccaccgcctgtcagcaacgcaacacactccgagtgggaaagagagggacaaaaaggttgacctctcgaaccaggccgccgttcgtgttgcgattctggtgtcgcatagccattgaggttgcaagcattcacggtccgatcgaccagggtccaaaaaagagttggggaagtcggcagtcttgttggatttgaccaccccttgctggccaattacctcgacgtccgcggcgagcgaacaccagcacggaaaggaaggcacacgaccgacagttgggatttgggaaagagtgtttctggacctggaattttggtctgaatattggctgtcaaattgactctgcggagctgctcctgccttccccccgcctgcaaacttgcgctagcattctgccaccctgacaaccttgttactggtaggtacctcgaggttgtcctcagctcccgtcaccaaaccgaccagacaaaaaaataccctcccctcccacctcctgctcccgtcaccaaaaccctccatttacctcatcttccgcgtttctttcttttccccttcaccccctcaagtcttgatccatcctcccccatttccctctttcttcctctttcaaccaactctcccatccctcaccatcaccacatcacctctccaaattcacttcctttataccccgccatcacaggaagtcattctctgggagagtcaccaattcacataaccacatttagtgaagatgccttccgcaactagcggccgcaagggaattatatagggattagggatggttagacatgggatcttttactttacggagcggttacaatcatgaatgttttcattttctttcttcggcaggtggtagagggtcatcaatgaagggaagggtgtatgacagaatacgtcattatcatatactggggaacttcccataatgcaatcaagacagcatcaactaccacgattggtgctatgtttccgttactgtgattgtcaacatccgggaattgcttttttatcgccaaatctctcacgtattggaataaagcaagtcagacggttccttgcccgtcgcccaaaagaccgctaaccgctaaccgccaagctgaccgagagacataccagcaatacggaacagcgagccacgaaacccgatgggaaggaccaaacccggctgagatttgagaccttctaccgacaatgttctatggattccgtcagcgacaaaccggagggtttcgctttccggactttgacggtgcacaagagatcgatgagctc

SEQ ID NO: 14 - DNA, 43 bp, Artificial sequence
  Description of the artificial sequence transition of the ppg1 promoter/ ppg1 terminator of the plasmid pSMY3
gaagatgcccatcgctagcggccgcaagcgctatgcctccccc

SEQ ID NO: 15 - DNA, 43 bp, Artificial sequence
  Description of the artificial sequence transition of the ppg1 promoter/ ppg1 terminator of the plasmid pSMY3; complementary strand
gggggaggcatagcgcttgcggccgctagcgatgggcatcttc

SEQ ID NO: 16 - protein, 13 aa, Artificial sequence
  Description of the artificial sequence amino acid sequence encoded by the transition sequence of the ppg1 promoter/ ppg1 terminator of the plasmid pSMY3
MPIASGRKRYASP

SEQ ID NO: 17 - DNA, 2729 bp, Artificial sequence
  Description of the artificial sequence nucleic acid sequence of the insert fragment of the plasmids pSMY3
gagctcgcgaatcctcccgaaacccacattcctcgatcttggttgacccaattctcattttcccgacatcgccaacaacctgtaacctacggcatcaactatgtgaaagcggttgatcaccccaaaggcatcgacactgaagacactgctagctgaaccttagctggattctgcgatactcatgaatggaagcacttgtcggcatctgtcatactcccgcacgaatatgataggtataccggctactggccaaacatgggttttcgtgggagcccaccaaggtaaactgaacagtagctcacaacaacaagagtgagcgatggacatgtatgctagaacaaccatggaacaagacatacactttccatggcgacagttggttcctaatgtccgccgctttggatggacaggcaatccgggaaaccagcaaacacaccttcttgtcccaaatgaaaggtgttgatcgcggagatacgaagatgagatgggcaaagatgggggaggcagagcgctcgctactgttatcaaagacgatcataaagccatggccaacaagacgaagaccacattgacgctggaataccagcgcttctgttactttttgtcctttgttgtgccaccttcgtccaacaccacctctgcgtctcctgcatgaaccgcacaaggacactctgatatcactcacccatctgactgcagcaattattttctagtattcctggtttccttgaggtactggatctcattccgaagaaaaggtggcgccaggggtgcagcctcgccaaaggctggcgctaggcgcaaaggagtcctttgagcaatcaaccacgctttcctaaaatggggttgaatagaacctcaataagcactttgttgctaacccctaaatttgccgccactcgaccacaccgcattgtccaagcaaccacggtgactcttttgttctgcccccggtctttattctagtaggacggtgcatctcacgtggcattaaagttaagcgcctcttgtgcatcctcatcttgacctcacccttgctttgtcttcctgcatcttaccttcttcagacccccggattctccccgagtgaccgtagcacccggctgcatgaagcaccctcgactactcggtcagttccgtcctgcaggccagacatcattgggcgagcaccgagtcccagcgagcttgacctgaacgtcttcatccgaacctcgatcttcgtgaaaccacaacgtttccgacacctcgggaccgagctttcatcccttcgaatcctcagcagtggcatttcatcccaactatcggagaatgtggctccatctccaggctccatgaaagccttcctctgaatagaggtctgaataaaccattgaccgatatgctctcgacctccaccaaagcaagcccttgatctcaataacgacaccatcacacgaataacaaacatgtcgctttcacgatctttgcttggttctggaccggccggtcgtcttctttaaccgaactccctccctcgtggccgccgccccttgaacgatcctggttgccaacaaggtttaccttcgattctatacagaacgcagttgactaaccaagccatcacagacttcgacagccgtgatagtttggatataaaagatggagttgatcacctcataattcatctttttttcctcaccacctttgacatcgccaatcaacccttcagaggtcttcattctctaaatcatcttaccattcttctttccaagccttctttcaagatgaagttcaccctccctcttgtcatcttcgccgccgtggcctccgccaccccggttgcccagcccatcgctgcggccgcaagcgctatgcctcccccgaggcggcttgcaacgcccccgacggctcttgcaccaaggccacccgtgacttgcacgccatgtacaacgtcgctcgtgccatcctcactgctcactctgatgagaactaggttagtttttacttcctcctcaaaccatcctacccaaccagatcacacatgtactaacccgcacatcctccttcagatttcccaccatgaaaatattcttcttctctcctgaaaagcaggcatgaccacaaaacaagaggggcaaaataaggcttggatataactttttgccaccctctttacaactttcacttctggaaaaagaaacatcacatacacatacacagaaaagcttttatggggttcttcacacacccttaacacataaccgggattggatatgtcgaataaacttgtacaatcctgtttttttatcaaatcaatcattcatcctccaccatgatgaggaccagcgggaaatgattgggggaagatatttgggaacgggcaatgtacttacactagctagtttctgatctgaccctttcagtatctcgaattcaaggacaaggctcgcttgctgattgatggcggccttgggtattttccttgacatgttcttctctccttggtgcagtgacttgcttctttctcgtgacatgttttcttgtttcatctgtgtttaggaatttgcgtgcgcattgcttggtgactctttgaaactctttttctgctgttactggttcggtgtttgaccgtcttaaggtatgaccatcaagctgtcggttgttgggtgagtactggcctaactgttgttttgaaatacagtttatacaagttattgcgtagtagaaggggatagcgatcaagaggatcc

SEQ ID NO: 18 - DNA, 55 bp, Artificial sequence
  Description of the artificial sequence transition of the acl1 promoter/lacZ gene in the plasmid pSMY1
gaagatgccttccgcaactagcggccgcgtcgttttacaacgtcgtgactgggaa

SEQ ID NO: 19 - DNA, 52 bp, Artificial sequence
  Description of the artificial sequence transition of the acl1-Promoter/lacZ gene in the plasmid pSMY1; complementary sequence
cagttggtctggtgtcaaaaataagcggccgcaagggaattatatagggatt

SEQ ID NO: 20 - DNA, 52 bp, Artificial sequence
  Description of the artificial sequence transition of the lacZ gene/acl1 terminator in the plasmid pSMY1
aatccctatataattcccttgcggccgcttatttttgacaccagaccaactg

SEQ ID NO: 21 - DNA, 55 bp, Artificial sequence
  Description of the artificial sequence transition of the lacZ gene/acl1 terminator in the plasmid pSMY1; complementary sequence
ttcccagtcacgacgttgaataacgacgcggccgctagttgcggaaggcatcttc

SEQ ID NO: 22 - protein, 17 aa, Artificial sequence
  Description of the artificial sequence amino acid sequence encoded by the transition sequence of the acl1 promoter/lacZ gene of the plasmid pSMY1
MPSATSGRVVLQRRDWN

SEQ ID NO: 23 - protein, 8 aa, Artificial sequence
  Description of the artificial sequence amino acid sequence encoded by the transition sequence of the lacZ gene / acl1 terminator of the plasmid pSMY1
QLVWCKQK

SEQ ID NO: 24 - DNA, 80 bp, Artificial sequence
  Description of the artificial sequence Linker 1
tcgacgcgcgcctcgagaggcctactagtgaattcagatctggatccgcggccgcatcgattcgcgaggtaccgcgcgca

SEQ ID NO: 25 - DNA, 80 bp, Artificial sequence
  Description of the artificial sequence Linker 2
gcgcgcggagctctccggatgatcacttaagtctagacctaggcgccggcgtagctaagcgctccatggcgcgcgttcga

SEQ ID NO: 26 - DNA, 21 bp, Artificial sequence
  Description of the artificial sequence Oligo 1095
cgccgtttcgtcggccacacc

SEQ ID NO: 27 - DNA, 21 bp, Artificial sequence
  Description of the artificial sequence Oligo 1096
cgcagagccagtagcggttgg

SEQ ID NO: 28 - DNA, 30 bp, Artificial sequence
  Description of the artificial sequence Oligo cpc9
ccatggttgcagttcctttctggttgatca

SEQ ID NO: 29 - DNA, 30 bp, Artificial sequence
  Description of the artificial sequence Oligo cpc11
ccatggagcatgattgttaatgcggagaag

SEQ ID NO: 30 - DNA, 30 bp, Artificial sequence
  Description of the artificial sequence Oligo cpc10
gatatcttgcagttcctttctggttgatca

SEQ ID NO: 31 - DNA, 30 bp, Artificial sequence
  Description of the artificial sequence Oligo cpc12
gatatcagcatgattgttaatgcggagaag

SEQ ID NO: 32 - DNA, 36 bp, Artificial sequence
  Description of the artificial sequence Oligo 1194
gagctcatcgatctcttgtgcaccgtcaaagtccgg

SEQ ID NO: 33 - DNA, 28 bp, Artificial sequence
  Description of the artificial sequence Oligo 1197
gcggccgctagttgcggaaggcatcttc

SEQ ID NO: 34 - DNA, 32 bp, Artificial sequence
  Description of the artificial sequence Oligo 1199
aagcttctagaatggcttgggctgctttggcc

SEQ ID NO: 35 - DNA, 32 bp, Artificial sequence
  Description of the artificial sequence Oligo 1200
gcggccgcaagggaattatatagggattaggg

SEQ ID NO: 36 - DNA, 33 bp, Artificial sequence
  Description of the artificial sequence Oligo 1206
gcggccgcttatttttgacaccagaccaactgg

SEQ ID NO: 37 - DNA, 33 bp, Artificial sequence
  Description of the artificial sequence Oligo 1215
gcggccgcgtcgttttacaactgctgtactggg

SEQ ID NO: 38 - DNA, 19 bp, Artificial sequence
  Description of the artificial sequence Oligo 1265
cccctcggttctacccgcc

SEQ ID NO: 39 - DNA, 19 bp, Artificial sequence
  Description of the artificial sequence Oligo 1266
gaaccagagggcaatctcc

SEQ ID NO: 40 - DNA, 26 bp, Artificial sequence
  Description of the artificial sequence Oligo ppg1-1
gagctcgcgaatcctcccgaaaccca

SEQ ID NO: 41 - DNA, 27 bp, Artificial sequence
  Description of the artificial sequence Oligo ppg1-2
gcggccgctagcgatgggctgggcaac

SEQ ID NO: 42 - DNA, 27 bp, Artificial sequence
  Description of the artificial sequence Oligo ppg1-3
gcggccgcaagcgctatgcctcccccg

SEQ ID NO: 43 - DNA, 27 bp, Artificial sequence
  Description of the artificial sequence Oligo ppg1-4
ggatcctcttgatcgctatccccttct

SEQ ID NO: 44 - DNA, 30 bp, Artificial sequence
  Description of the artificial sequence Oligo hsa1
gcggccgcaagtgggtaacctttatttccc

SEQ ID NO: 45 - DNA, 29 bp, Artificial sequence
  Description of the artificial sequence Oligo hsa2
gcggcgcttataagcctaaggcagcttga

SEQ ID NO: 46 - DNA, 27 bp, Artificial sequence
  Description of the artificial sequence Oligo ndk5-(SpeI)
atattactagtgtggtggtgcacctcg

SEQ ID NO: 47 - DNA, 29 bp, Artificial sequence
  Description of the artificial sequence Oligo ndk3-(EcoRI)
atattgaattctttgattatgggtggttc

SEQ ID NO: 48 - DNA, 26 bp, Artificial sequence
  Description of the artificial sequence Oligo cpc2-(AvaI)
gctcgagatcggaaggaggtggcgag

SEQ ID NO: 49 - DNA, 29 bp, Artificial sequence
  Description of the artificial sequence Oligo cpc2-(SpeI)
cactagtctttgcagttcctttctggttg

SEQ ID NO: 50 - DNA, 19 bp, Artificial sequence
  Description of the artificial sequence Oligo acl1-(NotI)
gcggccgcaagggaattat

SEQ ID NO: 51 - DNA, 20 bp, Artificial sequence
  Description of the artificial sequence Oligo acl1-(ClaI)
atcgatctcttgtgcaccgt

SEQ ID NO: 52 - DNA, 26 bp, Artificial sequence
  Description of the artificial sequence Oligo EGFP5′
cgaattcatggtgagcaagggcgagg

SEQ ID NO: 53 - DNA, 30 bp, Artificial sequence
  Description of the artificial sequence Oligo EGFP3′
cgaattctttacttgtacagctcgtccatg